(19) United States
(12) Patent Application Publication
(10) Pub. No.: US 2005/0239173 A1
(43) Pub. Date: Oct. 27, 2005

(54) Production of amino sugars

(75) Inventors: Sara C. McFarlan, St. Paul, MN (US); William A. Schroeder, Brooklyn Park, MN (US); Lawrence E. Fosdick, Oskalooska, IA (US); John A. Bohlmann, Ottumwa, IA (US)

Correspondence Address: CARGILL, INCORPORATED, LAW/24, 15407 MCGINTY ROAD WEST, WAYZATA, MN, 55391, US

(73) Assignee: Cargill, Inc.

(21) Appl. No.: 11/004,843

(22) Filed: Dec. 7, 2004

Related U.S. Application Data
(60) Provisional application No. 60/527,309, filed on Dec. 8, 2003.

(51) Int. Cl. C12P 19/28; C12N 1/18

(52) U.S. Cl. 435/85; 435/254.2

(57) ABSTRACT

The present invention provides a method for producing an amino sugar selected from the group consisting of N-acetylglucosamine, glucosamine, or a combination thereof. The method comprises culturing a yeast in a culture medium and recovering N-acetylglucosamine, glucosamine, or a combination thereof, wherein the yeast comprises an exogenous nucleic acid sequence encoding glucosamine-6-phosphate synthase operably linked to a promoter. The invention also provides a genetically modified yeast that produces an amino sugar selected from the group consisting of N-acetylglucosamine, glucosamine, or a combination thereof.

### BACKGROUND OF THE INVENTION

The present invention relates generally to the field of glucosamine and N-acetylglucosamine synthesis. Glucosamine, the 2-amino derivative of glucose, is a component of several biologically important polysaccharides. For example, a derivative of glucosamine, N-acetylmuramic acid is a prominent component of bacterial cell walls. Chitin is the principal structural constituent of the exoskeletons of invertebrates such as crustaceans, insects, and spiders and is also present in the cell walls of most fungi and many algae. This polysaccharide is a homopolymer of the glucosmine derivative N-acetylglucosamine.

Glucosamine is a key component of cartilage and is thought to be involved in joint function and repair. It has been tested in several scientific trials for treating osteoarthritis pain, rehabilitating cartilage, renewing synovial fluid, and repairing joints that have been damaged from osteoarthritis. Glucosamine has been shown to reduce the pain of osteoarthritis in some patients and improve joint structure. This compound and its derivatives, including N-acetylglucosamine, are sold as nutraceutical products for the treatment of osteoarthritic conditions in both humans and animals (“Glucosamine and Osteoarthritis”; Nutri-Chem™, Canada's Wellness Pharmacy; Booras C H (2000) “Glucosamine and Chondroiton for Osteoarthritis”, Jacksonville Medical Park Online).

Glucosamine is currently obtained by acid hydrolysis of chitin or acetylated chitosans. The raw material is often crustacean shells. Drawbacks of these methods are poor product yields, as well as limited supplies of raw materials. In addition there are food safety concerns due to the high incidence of allergic reactions to shellfish components by human consumers. There is a need in the industry for alternative methods for production of glucosamine and its derivative N-acetylglucosamine that are free of the drawbacks of the currently employed methods.

### SUMMARY OF THE INVENTION

It is an object of the invention to provide a method for producing an amino sugar selected from the group consisting of N-acetylglucosamine, glucosamine, or a combination thereof. The method comprises culturing a yeast in a culture medium and recovering N-acetylglucosamine, glucosamine, or a combination thereof, wherein the yeast contains an exogenous nucleic acid sequence encoding glucosamine-6-phosphate synthase operably linked to a promoter. The glucosamine-6-phosphate synthase enzyme transfers an amino group to fructose-6-phosphate.

Another object of the invention is to provide a method for producing glucosamine. The method involves culturing yeast in a culture medium, performing deacetylation, and recovering glucosamine, wherein the yeast contains an exogenous nucleic acid sequence encoding glucosamine-6-phosphate synthase operably linked to a promoter. In some embodiments, deacetylation involves contacting the culture medium with acid or an enzyme. In other embodiments, prior to performing deacetylation the yeast is separated from the culture medium and deacetylation is performed on the culture medium. In some embodiments, the enzyme is N-acetylglucosamine-6-phosphate deacetylase. In other embodiments, the N-acetylglucosamine-6-phosphate deacetylase is from the division Gammaproteobacteria. In other embodiments, the N-acetylglucosamine-6-phosphate deacetylase is from Escherichia coli. In some embodiments, the acid is selected from the group consisting of hydrochloric acid, sulfuric acid, nitric acid, nitrous acid, perchloric acid and phosphoric acid. In some embodiments, glucosamine is recovered by evaporative crystallization.

Another object of the invention is to provide a method for producing an amino sugar selected from the group consisting of N-acetylglucosamine, glucosamine, or a combination thereof. The method involves culturing a yeast in a culture medium and recovering N-acetylglucosamine, glucosamine, or a combination thereof, wherein (i) the yeast contains an exogenous nucleic acid sequence encoding glucosamine-6-phosphate synthase operably linked to a promoter, and (ii) the pH of the culture medium is equal to or less than pH 5.0. In some embodiments, the culture medium lacks an antimicrobial agent.

In some embodiments, the nucleic acid sequence encoding glucosamine-6-phosphate synthase contains a genetic modification which reduces feedback inhibition of the glucosamine-6-phosphate synthase. In other embodiments, the nucleic acid sequence encoding glucosamine-6-phosphate synthase encodes yeast glucosamine-6-phosphate synthase. In some embodiments, the yeast is Saccharomyces cerevisiae. In other embodiments, the yeast glucosamine-6-phosphate synthase gene is GFA1.

In some embodiments, the pH of the culture medium is equal to or less than pH5.0.

In other embodiments, the N-acetylglucosamine, glucosamine or combination thereof further comprises one or more carbohydrates.

In some embodiments, the yeast further contains one or more genetic modifications that minimize degradation of glucosamine-6-phosphate by the yeast. For example, the one or more genetic modification may be disruption of a nucleic acid sequence encoding a peptide selected from the group consisting of 6-phosphofructo-2-kinase, phosphoglucomutase, phosphofructokinase alpha subunit, phosphofructokinase beta subunit, N-acetylglucosamine-6-phosphate mutase, UDP N-acetylglucosamine-6-phosphate pyrophosphorylase, glucosamine-6-phosphate N-acetyltransferase, mannose-6-phosphate isomerase, hexokinase I and chitin synthase. In some embodiments the yeast is haploid and the one or more genetic modifications may be disruption of a nucleic acid sequence encoding a peptide selected from the group consisting of 6-phosphofructo-2-kinase, phosphoglucomutase, phosphofructokinase alpha subunit, phosphofructokinase beta subunit, hexokinase I and chitin synthase. In other embodiments, the yeast is heterozygous diploid and the one or more genetic modifications may be disruption of a nucleic acid sequence encoding a peptide selected from the group consisting of N-acetylglucosamine-6-phosphate mutase, UDP N-acetylglucosamine-6-phosphate pyrophosphorylase, glucosamine-6-phosphate N-acetyltransferase, mannose-6-phosphate isomerase, hexokinase I and chitin synthase. In other embodiments, the yeast is homozygous diploid and the one or more genetic modifications may be disruption of a nucleic acid sequence encoding a peptide selected from the group consisting of 6-phosphofructo-2-kinase, phosphoglucomutase, phosphofructokinase alpha subunit, phosphofructokinase beta subunit, hexokinase I and chitin synthase. In a preferred embodiment, the one or more genetic modification is disruption of a nucleic acid sequence encoding glucosamine-phosphate N-acetyltransferase in the yeast. In another preferred embodiment, the one or more genetic modification is disruption of a nucleic acid sequence encoding phosphoglucomutase. In another preferred embodiment, the one or more genetic modification is disruption of a nucleic acid sequence encoding UDP N-acetylglucosamine-6-phosphate pyrophosphorylase.

In some embodiments, the yeast is a MATa haploid strain having (i) an exogenous nucleic acid sequence encoding a-factor pheromone receptor and (ii) a genetic modification comprising disruption of a nucleic acid sequence encoding alpha-factor pheromone receptor. In other embodiments, the yeast is a MATalpha strain having (i) an exogenous nucleic acid sequence encoding alpha-factor pheromone receptor and (ii) a genetic modification comprising disruption of a nucleic acid sequence encoding a-factor pheromone receptor.

Another object of the invention is to provide a genetically modified yeast having (i) an exogenous nucleic acid sequence encoding glucosamine-6-phosphate synthase and (ii) one or more genetic modifications comprising disruption of a nucleic acid sequence encoding a peptide selected from the group consisting of 6-phosphofructo-2-kinase, phosphoglucomutase, phosphofructokinase alpha subunit, phosphofructokinase beta subunit, N-acetylglucosamine-6-phosphate mutase, UDP N-acetylglucosamine-6-phosphate pyrophosphorylase, glucosamine-6-phosphate N-acetyltransferase, mannose-6-phosphate isomerase, hexokinase I and chitin synthase. In some embodiments the yeast is diploid.

Another object of the invention is to provide a genetically modified yeast as described above in a culture medium of pH less than 5 containing an amino sugar selected from the group consisting of N-acetylglucosamine, glucosamine, or a combination thereof.

### BRIEF DESCRIPTION OF THE DRAWINGS

FIG. 1 shows a chromatogram of S. cerevisiae strain BY4743 culture medium samples withdrawn 90 hours after induction, demonstrating production of N-acetylgucosamine.

FIG. 2 shows a chromatogram of S. cerevisiae strain BY4743 culture medium samples withdrawn 90 hours after induction, demonstrating production of glucosamine.

### DETAILED DESCRIPTION OF THE PREFERRED EMBODIMENTS

The present invention provides a method for producing an amino sugar selected from the group consisting of N-acetylglucosamine, glucosamine, or a combination thereof.

The term “glucosamine” is used to mean the amino deoxysugar 2-amino-2-deoxyglucopyranose, which includes both enantiomers and salts thereof.

The term “N-acetylglucosamine” is used to mean the acetylated aminodeoxy sugar of glucosamine (2-acetamino-2-deoxy-D-glucose or 2-acetamino-2-deoxyglucopyranose), which can be in a monomer form, polymer, or an oligosacharide.

In some embodiments, the method involves culturing a yeast in a culture medium and recovering N-acetyl glucosamine, glucosamine, or a combination thereof. The yeast comprises an exogenous nucleic acid sequence encoding glucosamine-6-phosphate synthase operably linked to a promoter.

As used herein, the term “operably linked to a promoter” means that the nucleic acid sequence sought to be expressed and a regulatory nucleic acid sequence are connected in such a way as to permit expression of the nucleic acid sequence sought to be expressed.

The term “exogenous” as used herein with reference to nucleic acid and a particular cell refers to any nucleic acid that does not originate from that particular cell as found in nature. Thus, non-naturally-occurring nucleic acid is considered to be exogenous to a cell once introduced into the cell. It is important to note that non-naturally occurring nucleic acid can contain nucleic acid sequences or fragments of nucleic acid sequences that are found in nature provided the nucleic acid as a whole does not exist in nature. For example, a nucleic acid molecule containing a genomic DNA sequence within an expression vector is non-naturally-occurring nucleic acid, and thus is exogenous to a cell once introduced into the cell, since the nucleic acid molecule as a whole (genomic DNA plus vector DNA) does not exist in nature. Thus, any vector, autonomously replicating plasmid, or virus (e.g., retrovirus, adenovirus, or herpes virus) that as a whole does not exist in nature is considered to be non-naturally-occurring nucleic acid. It follows that genomic DNA fragments produced by PCR or restriction endonuclease treatment as well as cDNAs are considered to be non-naturally-occurring nucleic acid since they exist as separate molecules not found in nature. It also follows that any nucleic acid containing a promoter sequence and polypeptide-encoding sequence (i.e., cDNA or genomic DNA) in an arrangement not found in nature is non-naturally-occurring nucleic acid.

The exogenous nucleic acid sequence can be transfected into yeast using techniques that are well known to those of skill in the art. The transfected nucleic acid molecule can remain extrachromosomal or can integrate into one or more sites within a chromosome of the transfected (i.e., recombinant) host cell in such a manner that its ability to be expressed is retained.

The nucleic acid sequence encoding glucosamine-6-phosphate synthase can encode glucosamine-6-phosphate synthase from any organism. Preferably, the organism is yeast. More preferably, the yeast is Saccharomyces cerevisiae. In a preferred embodiment, the glucosamine-6-phosphate synthase gene is GFA1. The nucleotide sequence of the S. cerevisiae GFA1 gene is shown as SEQ ID NO: 1. The translated amino acid sequence of the S. cerevisiae GFA1 gene is shown as SEQ ID NO: 2. Saccharomyces cerevisiae nomenclature for naming genes, peptides and enzymes is used throughout this document except where otherwise noted.

In some embodiments, the nucleic acid sequence encoding glucosamine-6-phosphate synthase will preferably have at least 55% identity (more preferably at least 60%, more preferably at least 65%, more preferably at least 70%, more preferably at least 75%, more preferably at least 80%, more preferably at least 85%, more preferably at least 90%, more preferably at least 95%, and most preferably 99-100%) to the sequence of SEQ ID NO: 1. In other embodiments, the amino acid sequence of glucosamine-6-phosphate synthase will preferably have at least 55% identity (more preferably at least 60%, more preferably at least 65%, more preferably at least 70%, more preferably at least 75%, more preferably at least 80%, more preferably at least 85%, more preferably at least 90%, more preferably at least 95%, and most preferably 99-100%) to the sequence of SEQ ID NO:2. Those skilled in the art will recognize that several computer programs are available for determining sequence identity using standard parameters, for example Blast (Altschul, et al. (1997) Nucleic Acids Res. 25:3389-3402), Blast2 (Altschul, et al. (1990) J. mol. biol. 215:403-410), and Smith-Waterman (Smith, et al. (1981) J. Mol. Biol. 147:195-197).

In some embodiments, the nucleic acid sequence encoding the glucosamine-6-phosphate synthase contains at least 27, 30, 45, 60, 90 or 105 continuous nucleotides set forth in SEQ ID NO:1. In other embodiments, the amino acid sequence of glucosamine-6-phosphate synthase contains at least 9, 10, 15, 20, 30 or 35 contiguous amino acids of the full-length sequence set forth in SEQ ID NO:2.

It is an embodiment of the invention that the nucleic acid sequence encoding glucosamine 6-phosphate synthase comprises a genetic modification as compared to wild type glucosamine 6-phosphate synthase. The nucleic acid sequence encoding glucosamine 6-phosphate synthase comprising a genetic modification, thus, may encode a mutant or homolog glucosamine-6-phosphate synthase protein. The genetic modification can be achieved, for example, by mutation of the nucleic acid sequence encoding wild type glucosamine-6-phosphate synthase, e.g., by insertion, deletion, substitution, and/or inversion of nucleotides. Genetic modifications are described in detail below. The nucleic acid sequence encoding a mutant or homolog glucosamine-6-phosphate synthase protein can be produced and transformed into yeast using techniques that are well known to those of skill in the art. Exemplary techniques are disclosed, in Sambrook and Russell, 2001, Molecular Cloning A Laboratory Manual, 3^{rd} Edition, Cold Spring Harbor Laboratory Press, Cold Spring Harbor, N.Y. The genetic modification can be any genetic modification that enhances glucosamine-6-phosphate synthase activity. An enhancement in activity includes an increase in activity, stability, or substrate specificity. For example, it has been demonstrated that glucosamine-6-phosphate synthase activity is inhibited by glucosamine-6-phosphate. White, Biochem. J. 106:847-858 (1968). Thus, in one embodiment, the genetic modification reduces feedback inhibition of glucosamine-6-phosphate synthase activity (i.e., inhibition of glucosamine-6-phosphate activity by glucosamine-6-phosphate or a secondary product).

Example 5 describes two mutagenic protocols that can be used to increase the overall efficiency of enhancement of glucosamine-6-phosphate synthase activity. Activity of the GFA1 gene product (glucosamine-6-phosphate synthase/glutamine fructose-6-phosphate amidotransferase) can be enhanced through site-specific mutagenesis of the GFA1 gene using combinations of mutagenic primers encoding specific mutations or through error-prone PCR. Enhanced activity can be screened by complementation of hosts expressing the mutant GFA1 genes with glucosamine auxotrophs.

The culture medium includes assimilable sources of carbon, nitrogen and phosphate. The terms “culture medium” and “fermentation broth” are used throughout the specification interchangeably. One of ordinary skill in the art can readily determine the optimum culture medium for culturing a particular yeast. Exemplary culture mediums are provided in the Materials and Methods section of the Examples.

One of ordinary skill in the art can also readily determine the optimum culturing temperature for optimum growth of the yeast and production of N-acetylglucosamine, glucosamine, or a combination thereof. Exemplary culturing conditions are described in Examples 2 and 3.

The phrase “recovering N-acetylglucosamine, glucosamine or a combination thereof” refers simply to collecting the product from the culture medium and need not imply additional steps of separation or purification. For example, the step of recovering can refer to removing the entire culture (i.e., the yeast and the culture medium to recover both intracellular and extracellular product), removing the culture medium containing extracellular N-acetylglucosamine, glucosamine or a combination thereof, and/or removing the yeast containing intracellular N-acetylglucosamine, glucosamine or a combination thereof. These steps can be followed by further purification steps. For example, N-acetylglucosamine, glucosamine or a combination thereof can be recovered from the cell-free fermentation medium by conventional methods, such as chromatography, precipitation, extraction, crystallization (e.g., evaporative crystallization), membrane separation, dialysis, electrodialysis and reverse osmosis.

In some embodiments, N-acetylglucosamine, glucosamine or a combination thereof are recovered in substantially pure form. As used herein, “substantially pure” refers to a purity that allows for the effective use of the N-acetylglucosamine, glucosamine or a combination thereof as nutriceutical compounds for commercial sale. Typically, a “substantially pure” composition is at least about 95% N-acetylglucosamine, glucosamine or a combination thereof. Preferably, a “substantially pure” composition is at least about 98% N-acetylglucosamine, glucosamine or a combination thereof.

In other embodiments, N-acetylglucosamine, glucosamine or a combination thereof are recovered along with one or more carbohydrates. The carbohydrates may be derived either from the culture medium or from the yeast itself. Exemplary carbohydrates are selected from the group consisting of dextrose, xylose, mannose, N-acetyl-galactosamine, galactosamine, fructose, mannosamine, N-acetyl-mannosamine, and glucose.

Preferably, at least about 0.1 gram product (i.e., N-acetylglucosamine, glucosamine, or a combination thereof) per liter of culture medium is recovered from the yeast and/or culture medium. More preferably, by the method of the present invention, at least about 1 gram product (i.e., N-acetylglucosamine, glucosamine, or a combination thereof) per liter of culture medium is recovered, and even more preferably, at least about 5 grams product (i.e., N-acetylglucosamine, glucosamine, or a combination thereof) per liter of culture medium is recovered, and even more preferably, at least about 10 grams product (i.e., N-acetylglucosamine, glucosamine, or a combination thereof) per liter of culture medium is recovered, and even more preferably, at least about 20 grams product (i.e., N-acetylglucosamine, glucosamine, or a combination thereof) per liter of culture medium is recovered, and even more preferably, at least about 50 grams product (i.e., N-acetylglucosamine, glucosamine, or a combination thereof) per liter of culture medium is recovered, and even more preferably, at least about 100 grams product (i.e., N-acetylglucosamine, glucosamine, or a combination thereof) per liter of culture medium is recovered from the yeast and/or culture medium. In some embodiments, N-acetylglucosamine, glucosamine, or a combination thereof is recovered from the yeast and/or culture medium in an amount from about 0.1 gram product per liter of culture medium to about 100 grams product per liter of culture medium.

The term “about” will be understood by persons of ordinary skill in the art and will vary to some extent on the context in which it is used. If there are uses of the term which are not clear to persons of ordinary skill in the art given the context in which it is used, “about” will mean up to plus or minus 10% of the particular term.

Another embodiment of the present invention is a method of producing glucosamine comprising culturing yeast in a culture medium, performing deacetylation, and recovering glucosamine, wherein the yeast comprises an exogenous nucleic acid sequence encoding glucosamine-6-phosphate synthase operably linked to a promoter. In this embodiment, yeast comprising an exogenous nucleic acid sequence encoding glucosamine-6-phosphate synthase operably linked to a promoter is cultured in a culture medium, and subsequently, deacetylation is performed on the culture medium. In some embodiments, the culture medium comprises the yeast. In other embodiments, the yeast is separated from the culture medium. Any separation method can be employed to separate the yeast from the culture medium. Exemplary methods are centrifugation and filtration.

In some embodiments, deacetylation involves cleaving an N-acetyl group from N-acetylglucosamine. Deacetylation can be achieved by any method that achieves cleavage of an amide bond. In some embodiments, deacetylation comprises contacting the culture medium with an enzyme. The enzyme can be any enzyme that cleaves an amide bond, for example enzymes of the classification E. C. 3.5.-.- (non-peptidase amide carbon-nitrogen bond hydrolases). In some embodiments, the enzyme is N-acetylglucosamine-6-phosphate deacetylase.

Preferably, the N-acetylglucosamine-6-phosphate deacetylase is from the division Gammaproteobacteria. More preferably, the N-acetylglucosamine-6-phosphate deacetylase is from Escherichia coli, as described in Examples 10-13. The nucleic acid sequence of the Escherichia coli nagA gene is shown as SEQ ID NO: 3. The translated amino acid sequence of the Escherichia coli nagA gene is shown as SEQ ID NO: 4. In Example 12, N-acetylglucosamine was converted to glucosamine by enzymatic hydrolysis using the E. coli enzyme.

In some embodiments, the nucleic acid sequence encoding N-acetylglucosamine-6-phosphate deacetylase will preferably have at least 55% identity (more preferably at least 60%, more preferably at least 65%, more preferably at least 70%, more preferably at least 75%, more preferably at least 80%, more preferably at least 85%, more preferably at least 90%, more preferably at least 95%, and most preferably 99-100%) to the sequence of SEQ ID NO:3. In other embodiments, the amino acid sequence of N-acetylglucosamine-6-phosphate deacetylase will preferably have at least 55% identity (more preferably at least 60%, more preferably at least 65%, more preferably at least 70%, more preferably at least 75%, more preferably at least 80%, more preferably at least 85%, more preferably at least 90%, more preferably at least 95%, and most preferably 99-100%) to the sequence of SEQ ID NO:4. Those skilled in the art will recognize that several computer programs are available for determining sequence identity using standard parameters, for example Blast (Altschul, et al. (1997) Nucleic Acids Res. 25:3389-3402), Blast2 (Altschul, et al. (1990) J. mol. biol. 215:403-410), and Smith-Waterman (Smith, et al. (1981) J. Mol. Biol. 147:195-197).

In some embodiments, the nucleic acid sequence encoding the N-acetylglucosamine-6-phosphate deacetylase contains at least 27, 30, 45, 60, 90 or 105 continuous nucleotides set forth in SEQ ID NO:3. In other embodiments, the amino acid sequence of N-acetylglucosamine-6-phosphate deacetylase contains at least 9, 10, 15, 20, 30 or 35 contiguous amino acids of the full-length sequence set forth in SEQ ID NO:4.

In other embodiments, deacetylation comprises contacting the culture medium with acid. Exemplary acids are hydrochloric acid, sulfuric acid, nitric acid, nitrous acid, perchloric acid and phosphoric acid. The hydrolysis time depends on the acid concentration and temperature. A person of ordinary skill in the art can easily determine appropriate conditions for deacetylation by treatment of the culture medium with acid.

Following deacetylation, glucosamine is recovered, which is optionally followed by further purification steps, such as chromatography, precipitation, extraction, crystallization (e.g., evaporative crystallization), membrane separation, dialysis, electrodialysis and reverse osmosis. In a preferred embodiment, glucosamine is recovered by evaporative crystallization.

Example 7, describes a simulation of acid hydrolysis of N-acetylglucosamine in a test fermentation broth. More specifically, components that are typically found in a yeast fermentation broth were mixed together to provide a test broth. N-acetyl-glucosamine and various concentrations of acid were added to the broth. The resulting mixtures were incubated at various temperatures for various lengths of time. The results illustrate that glucosamine can be made via acid hydrolysis of N-acetyl-glucosamine in a fermentation broth.

Typically, in a commercial setting S. cerevisiae strains can be grown in culture medium and the yeast biomass can be separated from the fermentation broth prior to product recovery. In embodiments where N-acetylglucosamine is converted to glucosamine through acid hydrolysis, the hydrolysis step can be accomplished either before or after biomass separation. At the completion of the hydrolysis, the broth can then be evaporated to increase the concentrations of glucosamine hydrochloride and hydrochloric acid until the former exceeds its solubility. The hydrochloric acid concentration can reach 15-20% by weight during the evaporation stage. The solubility of glucosamine hydrochloride is soluble to approximately 3% by weight at this acid concentration. The crystals can be then harvested using a basket centrifuge and then dried. The hydrolysis time depends on the acid concentration and temperature. Excessive acid, temperature, or hydrolysis time may lead to a decrease of glucosamine and darkening of the broth.

Another embodiment is a method for producing an amino sugar selected from the group consisting of N-acetylglucosamine, glucosamine, or a combination thereof. The method comprises culturing a yeast in a culture medium and recovering N-acetylglucosamine, glucosamine, or a combination thereof, wherein (i) the yeast comprises an exogenous nucleic acid sequence encoding glucosamine-6-phosphate synthase operably linked to a promoter, and (ii) the pH of the culture medium is equal to or less than pH 5.0. A benefit to maintaining the pH at about pH 5 or less than about pH 5 is ease of sterility control. Sterility control of the culture medium is important for both small scale and large scale culturing. A culture medium having a pH of about 5 or less than about pH 5 is less likely to become contaminated with contaminating microorganisms because many microorganisms cannot survive in culture medium having a pH of about pH 5 or less than about pH 5. Moreover, yeast generally are not a phage sensitive as other microorganisms. In preferred embodiments, the pH is from about pH 2.5 to about pH 5.0. In some embodiments, the culture medium lacks an antimicrobrial agent. Example 4 shows that the parental strains of Examples 1-3 are not affected by concentrations of glucosamine as high as 20 g/L.

In an embodiment of the invention, the yeast employed in the method for producing N-acetylglucosamine, glucosamine or a combination thereof, which comprises an exogenous nucleic acid sequence encoding glucosamine-6-phosphate synthase operably linked to a promoter, further comprises one or more genetic modifications that minimize degradation of glucosamine-6-phosphate by the yeast. As used herein, a genetically modified yeast has a genome which is modified (i.e., mutated or changed) from its normal (i.e., wild-type or naturally occurring) form. Genetic modification of a yeast can be accomplished using classical strain development and/or molecular genetic techniques. Such techniques are generally disclosed, for example, in Wach A, Brachat A, Pohlmann R and Philippsen P. (1994). New heterologous modules for classical or PCR-based gene disruptions in Saccharomyces cerevisiae. Yeast. 10: 1793-1808; Goldstein A L and McCusker J H. (1999). Three New Dominant Drug Resistance Cassettes for Gene Disruption in Saccharomyces cerevisiae. Yeast. 15: 1541-1553.

Techniques for genetic modification of a microorganism are described in detail in the Examples section. See, e.g., Examples 1, 5, 6 and 8. A genetically modified yeast can include a natural genetic variant as well as a yeast in which nucleic acid molecules have been inserted, deleted or modified (i.e., mutated; e.g., by insertion, deletion, substitution, and/or inversion of nucleotides), in such a manner that such modifications provide the desired effect within the yeast. As used herein, the term “disrupt” is meant to include any genetic modification which impairs gene expression or function.

According to the present invention, a genetically modified yeast includes a yeast that has been modified using recombinant technology. The genetic modification may result in a decrease in gene expression, in the function of the gene, or in the function of the gene product (i.e., the protein encoded by the gene) and may involve inactivation (complete or partial), deletion, interruption, blockage or down-regulation of a gene. For example, a genetic modification in a gene which results in a decrease in the function of the protein encoded by such gene, can be the result of a complete deletion of the gene (i.e., the gene does not exist, and therefore the protein does not exist), a mutation in the gene which results in incomplete or no translation of the protein (e.g., the protein is not expressed), or a mutation in the gene which decreases or abolishes the natural function of the protein (e.g., a protein is expressed which has decreased or no enzymatic activity or action). Genetic modifications which result in an increase in gene expression or function may be a result of amplification, overproduction, overexpression, activation, enhancement, addition, or up-regulation of a gene.

The yeast may comprise any genetic modification(s) that minimize degradation of glucosamine-6-phosphate by the yeast. Preferred genetic modification(s) comprise disruption of a nucleic acid encoding a peptide involved in catabolism of glucosamine and N-acetylglucosamine. Exemplary peptides are 6-phosphofructo-2-kinase, phosphoglucomutase, phosphofructokinase alpha subunit, phosphofructokinase beta subunit, N-acetylglucosamine-6-phosphate mutase, UDP N-acetylglucosamine-6-phosphate pyrophosphorylase, glucosamine-6-phosphate N-acetyltransferase, mannose-6-phosphate isomerase, hexokinase I and chitin synthase. Phosphoglucomutase includes a number of isozymes. Chitin synthase includes several forms, such as chitin synthase 1, 2 and 3. A preferred chitin synthase is chitin synthase 3. The yeast employed in the inventive method may comprise a genetic modification of one or more of these enzymes.

In a preferred embodiment, the yeast comprises one or more genetic modifications comprising disruption of a nucleic acid sequence selected from the group of genes coding for the following enzymes: glucosamine-6-phosphate N-acetyltransferase, phosphoglucomutase and UDP N-acetylglucosamine-6-phosphate pyrophosphorylase. Example 6 illustrates a method for disruption of the gene encoding UDP N-acetylglucosamine-6-phosphate pyrophosphorylase in a homozygous diploid mutant of the PGM2 gene (encodes an isozyme of phosphoglucomutase). The resulting mutant is a particularly suitable host for overexpression of the GFA1 gene and high level production of N-acetylglucosamine.

In addition, Example 9 illustrates a method for producing glucosamine which involves disrupting the gene encoding glucosamine-6-phosphate acetyltransferase in the homozygous host described in Example 6. Overexpression of the GFA1 gene in this strain should result in high level production of glucosamine. Examples 10, 11 and 13 also describe methods for production of glucosamine by simultaneous overexpression of the GFA1 gene and the E. coli nagA gene in a suitable yeast host such as the homozygous diploid mutant of the PGM2 gene. The use of yeast to produce glucosamine provides the added advantage of having a culture medium that is at a relatively low pH (for example less that pH 5). The lower pH of a yeast media provides a more stable environment for the glucosamine, especially when compared to culture mediums used to incubate other non-yeast microorganisms which are typically in the range of pH 6-8.

A feature of using yeast in the novel method of production is that the yeast can be haploid or diploid. As used herein, the term “diploid” includes heterozygous diploid and homozygous diploid. In some instances, the null mutation is not viable. In these cases, it is possible to utilize a heterozygous diploid yeast in which one copy of the gene is disrupted and the other copy of the gene encodes an active enzyme. Thus, the yeast can be haploid, homozygous diploid or heterozygous diploid.

In some embodiments, the genetically modified yeast is haploid and the one or more genetic modifications comprises disruption of a nucleic acid sequence encoding a peptide selected from the group consisting of 6-phosphofructo-2-kinase, phosphoglucomutase, phosphofructokinase alpha subunit, phosphofructokinase beta subunit, hexokinase I and chitin synthase.

In other embodiments the genetically modified yeast is heterozygous diploid and the one or more genetic modifications comprises disruption of a nucleic acid sequence encoding a peptide selected from the group consisting of N-acetylglucosamine-6-phosphate mutase, UDP N-acetylglucosamine-6-phosphate pyrophosphorylase, glucosamine-6-phosphate N-acetyltransferase, mannose-6-phosphate isomerase, hexokinase I and chitin synthase.

In other embodiments, the genetically modified yeast is homozygous diploid and the one or more genetic modifications comprises disruption of a nucleic acid sequence encoding a peptide selected from the group consisting of 6-phosphofructo-2-kinase, phosphoglucomutase, phosphofructokinase alpha subunit, phosphofructokinase beta subunit, hexokinase I and chitin synthase.

Examples 1-3 illustrate production of N-acetylglucosamine, glucosamine, or a combination thereof by yeast comprising an exogenous nucleic acid sequence encoding glucosamine-6-phosphate synthase operably linked to a promoter and further comprising a genetic modification comprising disruption of a nucleic acid sequence encoding a peptide involved in N-acetylglucosamine or glucosamine catabolism. The GFA1 gene encoding the enzyme glucosamine-6-phosphate synthase was cloned into three yeast expression vectors and expressed in eleven Saccharomyces cerevisiae diploid and haploid strains. Nine of these strains have single deletions of genes that are known to be involved in the catabolism of glucosamine and N-acetylglucosmine in yeast. All were able to grow in a defined medium adjusted to pH 4 and, in fact, the pH dropped from the starting pH to 3.5 or less within 12 hours after inoculation. Strains grown and induced under the same conditions transformed with the yeast plasmid(s) lacking the GFA1 gene(s) secreted little or no N-acetylglucosamine and no detectable levels of glucosamine.

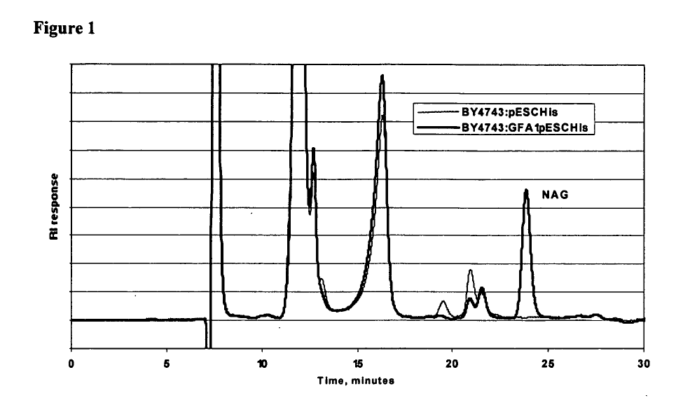

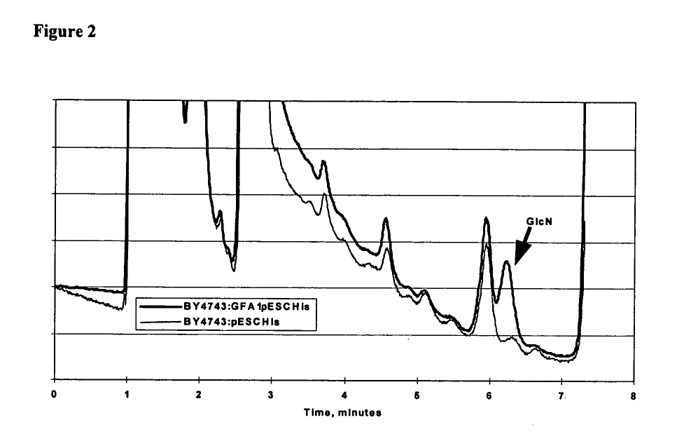

The haploid strain with a deletion in the gene encoding a phosphoglucomutase isozyme secreted more than 350 mg/L (˜1.6 mM) of N-acetylglucosamine 90 h after induction of GFA1 gene. By comparison the parental haploid strain secreted about 150 mg/L (˜0.7 mM) of the same product. The diploid strain that synthesized the highest amount of this product has a deletion in one of its genes encoding UDP-N-acetylglucosamine pyrophosphorylase (˜150 mg/L of secreted product). The corresponding parental strain secreted at least one-third less product per liter. This strain was also analyzed for glucosamine production and was found to secrete about 0.7 mg/L of the non-acetylated derivative. FIGS. 1 and 2 are chromatograms showing production of glucosamine and N-acetylglucosamine by S. cerevisiae strain BY4743 90 hours after induction.

Chitin is a polymer of N-acetylglucosamine. Hence, another embodiment involves metabolic engineering to increase glucosamine and/or N-acetylglucosamine biosynthesis by genetically modifying the yeast to signal for increased chitin biosynthesis but to divert the resulting flow of precursor (glucosamine or N-acetylglucosamine) into free, excreted monomer, before chitin is produced. Thus, in one embodiment of the novel method for producing N-acetylglucosamine, glucosamine or a combination thereof, the yeast is a MATa haploid strain comprising (i) an exogenous nucleic acid sequence encoding a-factor pheromone receptor and (ii) a genetic modification comprising disruption of a nucleic acid sequence encoding alpha-factor pheromone receptor. In another embodiment, the yeast is a MATalpha strain comprising (i) an exogenous nucleic acid sequence encoding alpha-factor pheromone receptor and (ii) a genetic modification comprising disruption of a nucleic acid sequence encoding a-factor pheromone receptor.

This embodiment is illustrated in Example 8. In S. cerevisiae, the STE3 gene encodes the a-factor pheromone receptor. The nucleotide sequence of the S. Cerevisiae STE3 gene is shown in SEQ ID NO: 5. The translated amino acid sequence of the S. Cerevisiae STE3 gene is shown in SEQ ID NO: 6.

In some embodiments, the nucleic acid sequence encoding the a-factor pheromone receptor will preferably have at least 55% identity (more preferably at least 60%, more preferably at least 65%, more preferably at least 70%, more preferably at least 75%, more preferably at least 80%, more preferably at least 85%, more preferably at least 90%, more preferably at least 95%, and most preferably 99-100%) to the sequence of SEQ ID NO:5. In other embodiments, the amino acid sequence of the a-factor pheromone receptor will preferably have at least 55% identity (more preferably at least 60%, more preferably at least 65%, more preferably at least 70%, more preferably at least 75%, more preferably at least 80%, more preferably at least 85%, more preferably at least 90%, more preferably at least 95%, and most preferably 99-100%) to the sequence of SEQ ID NO:6. Sequences for alpha-factor pheromone receptor are known in the art. Those skilled in the art will recognize that several computer programs are available for determining sequence identity using standard parameters, for example Blast (Altschul, et al. (1997) Nucleic Acids Res. 25:3389-3402), Blast2 (Altschul, et al. (1990) J. mol. biol. 215:403-410), and Smith-Waterman (Smith, et al. (1981) J. Mol. Biol. 147:195-197).

In some embodiments, the nucleic acid sequence encoding the a-factor pheromone receptor contains at least 27, 30, 45, 60, 90 or 105 continuous nucleotides set forth in SEQ ID NO:5. In other embodiments, the amino acid sequence of a-factor pheromone receptor contains at least 9, 10, 15, 20, 30 or 35 contiguous amino acids of the full-length sequence set forth in SEQ ID NO:6.

The onset of mating is signaled when the a-factor pheromone receptor is bound by a-factor pheromone produced by MATa cells. Haploid strains exposed to their complimentary mating factor pheromone (a-cells exposed to alpha-factor and vice versa) have been shown to increase chitin biosynthesis. Overexpression of the STE3 gene in a host organism lacking the mating type alpha-factor pheromone receptor (STE2 deletion) and producing its own a-factor pheromone can be expected to self-induce chitin biosynthesis.

Other embodiments provide genetically modified yeast as described above. In an exemplary embodiment, the genetically modified yeast comprises (i) an exogenous nucleic acid sequence encoding glucosamine-6-phosphate synthase and (ii) one or more genetic modifications comprising disruption of a nucleic acid sequence encoding a peptide selected from the group consisting of 6-phosphofructo-2-kinase, phosphoglucomutase, phosphofructokinase alpha subunit, phosphofructokinase beta subunit, N-acetylglucosamine-6-phosphate mutase, UDP N-acetylglucosamine-6-phosphate pyrophosphorylase, glucosamine-6-phosphate N-acetyltransferase, mannose-6-phosphate isomerase, hexokinase I and chitin synthase. Such a yeast is described above. In some embodiments the yeast is diploid (homozygous or heterozygous).

The following non-limiting examples are given by way of illustration only and are not to be considered limitations of this invention. There are many apparent variations within the scope of this invention.

### EXAMPLES

Materials and Methods

E. coli DH10B ElectroMAX cells were purchased from Invitrogen Life Technologies, Inc (Carlsbad, Calif.). E. coli BL21(DE3) cells were from Novagen (Madison, Wis.). Saccharomyces cerevisiae genomic DNA was from ResGen Invitrogen, Corp (Huntsville, Ala.). S. cerevisiae strains were from Invitrogen (Brachmann C B, Davies A, Cost G J, Caputo E, Li J, Hieter P, Boeke J D (1988) Designer deletion strains derived from Saccharomyces cerevisiae S288C: a useful set of strains and plasmids for PCR-mediated gene disruption and other applications. Yeast 14(2):115-32). The pESC Yeast Epitope Tagging Vector System (Stratagene, La Jolla, Calif.) was used to clone and express the Saccharomyces cerevisiae GFA1 gene into S. cerevisiae strains. The pESC vectors contain both the GAL1 and the GAL10 promoters on opposite strands, with two distinct multiple cloning sites, allowing for simultaneous expression of two genes. These promoters are repressed by glucose and induced by galactose. The pESC plasmids are shuttle vectors, allowing the initial construct to be made in E. coli (with the bla gene for selection on 100 ug/ml Ampicillin); however, no bacterial ribosome binding sites are present in the multiple cloning sites. Polymerase chain reactions were carried out using an Opti-Prime PCR Optimization Kit (Stratagene) or Expand DNA polymerase (Roche Molecular Biochemicals; Indianapolis, Ind.). Plasmid DNA was purified from bacterial cells using a QIAprep Spin Miniprep kit (Qiagen, Valencia, Calif.) while plasmid DNA was purified from yeast cells with a Zymoprep Yeast Plasmid Miniprep kit (Zymo Research, Orange, Calif.). The Rapid DNA Ligation Kit was from Roche Diagnostics Corp (Indianapolis, Ind.). The QIAQuick Gel Purification and PCR Purification kits were purchased from Qiagen. The S.c. EasyComp™ Transformation Kit was from Invitrogen Corp (Carlsbad, Calif.). Microbial growth media components were from Becton Dickinson Microbiology Systems (Sparks, Md.) or VWR Scientific Products (So. Plainfield, N.J.), and other reagents were of analytical grade or the highest grade commercially available. Primers were purchased from Integrated DNA Technologies, Inc. Restriction enzymes were from New England Biolabs, Inc (Beverly, Mass.). Electrophoresis of DNA samples was carried out using a Bio-Rad Mini-Sub Cell GT system (DNA) (Bio-Rad Laboratories, Hercules, Calif.) while protein samples were analyzed using a Bio-Rad Protein 3 mini-gel system and precast 4-15% gradient SDS-PAGE gels. An Eppendorf Mastercycler Gradient thermal cycler was used for PCR experiments. UV-visible spectrometry was done using a Molecular Devices SpectraMAX Plus spectrophotometer (Sunnyvale, Calif.). Electroporations of DNA samples were performed using a Bio-Rad Gene Pulser II system while protein samples were analyzed using a Bio-Rad Protein 3 mini-gel system and precast 4-15% gradient SDS-PAGE gels. Automated DNA sequencing was carried by SeqWright (Houston, Tex.) using the dideoxynucleotide chain-termination DNA sequencing method.

Recombinant DNA techniques for PCR, purification of DNA, ligations and transformations were carried out according to established procedures (Sambrook and Russell, 2001, Molecular Cloning A Laboratory Manual, 3^{rd} Edition, Cold Spring Harbor Laboratory Press, Cold Spring Harbor, N.Y.).

Medium Recipes

LB Medium (Miller)

Per liter

| | | |
|---|---|---|
| 10 | g | tryptone |
| 5 | g | yeast extract |
| 10 | g | sodium chloride |

autoclave for 15 min at 121° C.
(for solid medium add 1.5% agar before autoclaving)
   2xYT Medium

Per liter

| | | |
|---|---|---|
| 16 | g | tryptone |
| 10 | g | yeast extract |
| 5 | g | sodium chloride |

autoclave for 15 min at 121° C.
   SC-His Defined Medium

Per liter

Dissolve in water to 800 mL

| | | |
|---|---|---|
| 6.7 | g | Yeast Nitrogen Base without amino acids (Difco) |
| 0.1 | g | adenine |
| 0.1 | g | uracil |
| 0.1 | g | arginine |
| 0.1 | g | cysteine |
| 0.1 | g | lysine |
| 0.1 | g | threonine |
| 0.05 | g | aspartic acid |
| 0.05 | g | isoleucine |
| 0.05 | g | leucine |
| 0.05 | g | methionine |
| 0.05 | g | phenylalanine |
| 0.05 | g | proline |
| 0.05 | g | serine |
| 0.05 | g | tyrosine |
| 0.05 | g | tryptophan |
| 0.05 | g | valine |

autoclave for 15 min at 121° C., cool
   SC-His Defined Medium

Per liter

Dissolve in water to 800 mL

| | | |
|---|---|---|
| 6.7 | g | Yeast Nitrogen Base without amino acids (Difco) |
| 0.1 | g | adenine |
| 0.1 | g | uracil |
| 0.1 | g | arginine |
| 0.1 | g | cysteine |
| 0.1 | g | lysine |
| 0.1 | g | threonine |
| 0.05 | g | aspartic acid |
| 0.05 | g | isoleucine |
| 0.05 | g | leucine |
| 0.05 | g | methionine |
| 0.05 | g | phenylalanine |
| 0.05 | g | proline |
| 0.05 | g | serine |
| 0.05 | g | tyrosine |
| 0.05 | g | tryptophan |
| 0.05 | g | valine |

autoclave for 15 min at 121° C., cool
   SC-His-Trp-Leu Defined Medium

Per liter

Dissolve in water to 800 mL

| | | |
|---|---|---|
| 6.7 | g | Yeast Nitrogen Base without amino acids (Difco) |
| 0.1 | g | adenine |
| 0.1 | g | uracil |
| 0.1 | g | arginine |
| 0.1 | g | cysteine |
| 0.1 | g | lysine |
| 0.1 | g | threonine |
| 0.05 | g | aspartic acid |
| 0.05 | g | isoleucine |
| 0.05 | g | methionine |
| 0.05 | g | phenylalanine |
| 0.05 | g | proline |
| 0.05 | g | serine |
| 0.05 | g | tyrosine |
| 0.05 | g | valine |

autoclave for 15 min at 121° C., cool
   Assay Methods Used To Determine Glucosamine And N-Acetylglucosamine Formation In Fermentation Broth And Cell Extract Samples.

Elson and Morgan Assay

Fermentation broth and cell extract samples were routinely assayed using the method of Elson and Morgan (Biochem. J. 27:1824-1828) described by Zalkin (1985, Method Enzymol. 113:278-281) and Roden et al. (1997, Anal. Biochem. 254:240-248). Typically, to 0.08 mL of fermentation broth or 0.015 to 0.02 mL of cell extract was added 0.01 mL of saturated sodium bicarbonate solution and 0.01 mL of cold, freshly prepared 5% aqueous acetic anhydride. After a 3 minute incubation at room temperature, the mixture was incubated at 100° C. for 3 min to drive off the excess acetic anhydride. After cooling to room temperature, 0.12 mL of potassium borate, pH 9.2, was added and the mixture was incubated at 100° C. for 3 min. After cooling to room temperature, 1.0 mL of Ehrlich's reagent (1 gram p-dimethylaminobenzaldehyde in 100 mL glacial acetic acid containing 0.125 M HCl) was added to each tube. The tubes were incubated at 37° C. for 20 min and the absorbance at 585 nm was measured. All samples were assayed in duplicate. A standard curve was generated using 0.005 to 0.1 μmole glucosamine for quantification of product formation. Samples were assayed with and without the acetic anhydride addition to determine the amounts of glucosamine and N-acetylglucosamine present in the sample. The amount of N-acetylglucosamine was calculated from the results of assays without the acetic anhydride addition. The amount of glucosamine was calculated as the difference between the results with and without the acetic anhydride addition.

High Performance Liquid Chromatography Method I

N-acetylglucosamine (NAG) was determined using high performance liquid chromatography (HPLC) with a combination of refractive index and UV (195 nm) detection. The system comprised a SIL-10AXL autosampler, SCL-10AVP controller, LC-10AT pump, CTO-6A column oven, SPD-M10AVP diode-array detector, and a RID-6A refractive index detector, all from Shimadzu Scientific Instruments, Inc., Columbia, Md., U.S.A. The column was a MetaCarb H Plus, 300×7.8 mm, from Varian, Inc., Torrence, Calif., U.S.A. The eluent was 0.01N sulfuric acid in water; the flow rate was 0.4 mL/min. The column was maintained at 70° C. Broth samples were analyzed neat after filtration through 0.2μ nylon filters. NAG eluted at 23.9 minutes and was well resolved from other species in the samples. Multiple standards confirmed good linearity over the concentration range of interest. The UV spectrum from 190 to 350 nm indicated no measurable co-eluting peaks, and the retention time and ratio of responses between the detectors confirmed the identity of NAG.

High Performance Chromatography Method II

The free glucosamine (GlcN) in fermentation broth samples was also determined using high performance anion-exchange chromatography with pulsed amperometric detection (HPAEC-PAD). The system consisted of an EG40 eluent generator, GP50 gradient pump, AS40 autosampler, LC25 column oven, and ED40 electrochemical detector, all produced by Dionex Corporation, Sunnyvale, Calif., U.S.A. The method was adapted from Dionex Corporation Technical Note 40. A Dionex CarboPac PA-20 column was used in place of the PA-10 described in the Technical Note. The eluent was 8 mM KOH at 0.5 mL/min. The column and detector were maintained at 30° C. The injection volume was 10 μL. The standard was glucosamine hydrochloride at 10.8 mg/L. Fermentation broth samples were diluted five-fold with deionized water, ASTM Type II, and filtered through 0.2μ vial filters in the autosampler. Multiple standards were analyzed before each sample set. Standards and blanks were measured before and after each injection to verify that retention times were not affected by other broth components, and that carryover between injections was minimized. The glucosamine retention time did not vary by more than 0.01 minutes during the experimental sample set.

### Example 1

### Cloning Of The GFA1 Gene into S. cerevisiae Strains

The nucleic acid sequence of GFA1 (coding for glucosamine-6-phosphate synthase), SEQ ID NO: 1, was obtained from the Stanford yeast genome database. The GFA1 gene was cloned into the pESChis, pESCtrp, and pESCleu vectors singly behind the Gal1 promoter or behind both the Gal1 and Gal10 promoters. Primers for the synthesis of the gene with appropriate restriction sequences for the pESC vectors 5‘of the gene’s ATG start codon and 3‘of each gene’s stop codon were designed for PCR amplification using S. cerevisiae genomic DNA as template.

Forward primer for GFA1 with BamHI site:
5′- CGCGGATCCAGAATGTGTGGTATCTTTGG - 3′
Reverse primer for GFA1 with XhoI site:
5′- CCGCTCGAGTTATTCGACGGTAACAGATTTAGC - 3′
Forward primer for GFA1 with SpeI site:
5′- GGACTAGTATGTGTGGTATCTTTGGTTACTGC - 3′
Reverse primer for GFA1 with SacI site:
5′- CCGGAGCTCTTATTCGACGGTAACAGATTTAGC - 3′

Note: Italics indicate the restriction sites while bold lettering indicates the start and stop codons.

Construction of GFA1pESCHis, GFA1pESTrp, and GFA1pESCLeu

The GFA1 gene was amplified by PCR using the primers with BamHI and XhoI restriction sites. The thermocycler program used included a hot start at 96° C. for 5 min; and 30 repetitions of the following steps: 94° C. for 30 sec, 60° C. for 1 min, and 72° C. for 1 min, 30 sec. After the 30 cycles the sample was incubated at 72° C. for 7 min and then stored at 4° C. The PCR product was purified from a 1% TAE-agarose gel (QIAQuick Gel Purification kit) and after restriction digestion of both the PCR product and the pESCHis vector with BamHI and XhoI, the ligation was carried out using the Rapid DNA Ligation Kit (Roche). The ligation mixture was desalted and then transformed into E. coli DH 10B ElectroMAX cells using the BioRad recommended procedure for transformation of E. coli cells with 0.2 cm micro-electroporation cuvettes. After recovery in SOC medium the transformation mixture was plated on LB plates containing ampicillin at 100 μg/mL. Plasmid DNA was isolated from liquid cultures [5 mL 2xYT medium+ampicillin (100 μg/mL) grown overnight at 37° C.] of colonies picked from the LB+ampicillin (100 μg/mL) plates and purified. The plasmids were then screened by restriction digestion and the sequences were verified by dideoxynucleotide chain-termination DNA sequencing.

The plasmid DNA from a GFA1pESCHis clone with the correct insert sequence as well as plasmids pESCTrp and pESCLeu were digested with BamHI and XhoI. The 2.1 Kbp band carrying the GFA1 gene and the linear pESCTrp and pESCLeu plasmids were purified from a 1% TAE-agarose gel and ligated as described above. After removing the salts and proteins using a QIAQuick PCR Clean-up kit, the ligation mixtures were transformed into E. coli DH10B cells. Plasmid DNA was purified from ampicillin resistant cells and screened by restriction digestion.

Construction of 2(GFA1)pESCHis, 2(GFA1)pESTrp, and 2(GFA1)pESCLeu

The GFA1 gene was amplified by PCR using the primers with SpeI and SacI restriction sites. The PCR product was purified from a 1% TAE-agarose gel (QIAQuick Gel Purification Kit) and the sequence was verified by dideoxynucleotide chain-termination DNA sequencing. The GFA1pESCHis, GFA1pESCTrp, and GFA1pESCLeu plasmids and the PCR product were digested with SpeI and SacI. The plasmids were purified from a 1% TAE-agarose gel while the restriction digest mixture of the PCR product was purified using a QIAQuick PCR Clean-up kit. Ligations and transformations into E. coli DH10B cells were carried out as described above. Plasmid DNA was purified from ampicillin resistant cells and screened by restriction digestion. Plasmids carrying two copies of the GFA1 gene were chosen for transformation into the S. cerevisiae strains.

Competent cells of the S. cerevisiae strains listed below were prepared using an S.c. EasyComp™ Transformation Kit (Invitrogen Corp). Aliquots (50 μL) were frozen at −80° C. and thawed just prior to use.

S. cerevisiae Strains

| | |
|---|---|
| BY4742 | haploid parental strain (MATαhis3-Δ1, leu2-Δ0, |
| | lys2-Δ0, ura3-Δ0) |
| 12266 | PFK26 (YIL107C) deletion (6-phosphofructo-2-kinase) |
| | (haploid) |
| 16545 | PGM2 (YMR105C) deletion (phosphoglucomutase isozyme) |
| | (haploid) |
| 14977 | PGM1 (YKL127W) deletion (phosphoglucomutase minor form) |
| | (haploid) |
| 15893 | PFK1 (YGR240C) deletion (phosphofructokinase alpha |
| | subunit) (haploid) |
| 10791 | PFK2 (YMR205C) (phosphofructokinase beta subunit) |
| | (haploid) |
| BY4743 | diploid parental strain (MATa/αhis3-Δ1/his3-Δ1, |
| | leu2-Δ0/leu2-Δ0, met15-Δ0/MET15⁺, LYS2⁺/ |
| | lys2-Δ0, ura3-Δ0/ura3-Δ0) |
| 20299 | PCM1 (YEL058W) deletion (NAcglucosamine-6-P mutase) |
| | (heterozygous diploid) |
| 23800 | QRI1, UAP1 (YDL103C) deletion (UDP-NAc-glucosamine |
| | pyrophosphorylase) heterozygousdiploid) |
| 25635 | GNA1, PAT1 (YFL017C) deletion (glucosamine-6- |
| | phosphate N-acetyltransferase) (heterozygous diploid) |
| 20324 | PMI, PMI40 (YER003C) deletion (mannose-6-phosphate |
| | isomerase) heterozygous diploid) |

Transformations of the pESC vector constructs into S. cerevisiae competent cells were also carried out using the S.c. EasyComp™ Transformation Kit. The vectors pESCHis or GFA1pESCHis were transformed singly into each strain. A 100 μL aliquot from each transformation reaction was spread on SC-His plates (medium recipes from Stratagene pESC manual). The plate medium of the strains with single gene deletions also contained 0.2 mg/mL geneticin. The plates were incubated for 2 days at 30° C. Colonies from each plate were used to inoculate 5 mL liquid cultures of SC-His medium. The cultures were incubated overnight at 30 C and the cells were harvested by centrifugation, and plasmid DNA was isolated from the cells using a Zymoprep Yeast Plasmid Miniprep kit. After analysis of the isolated DNA by PCR, one isolate from each construct that generated the predicted PCR products was chosen for expression studies.

The three vectors pESCHis, pESCTrp, and pESCHis or the three vectors 2(GFA1)pESCHis, 2(GFA1)pESCTrp, 2(GFA1)pESCLeu were simultaneously transformed into each S. cerevisiae strain as described above and the transformation mixtures were plated on SC-His-Trp-Leu plates. The plate medium of the strains carrying single gene deletions also contained 0.2 mg/mL geneticin. After analysis by PCR, one isolate carrying the multiple plasmids without the GFA1 inserts and one isolate carrying the plasmids with the GFA1 gene inserted downstream of both the GAL1 and GAL10 promoters were chosen for expression studies. No transformants carrying the multiple plasmids with GFA1 insert were detected with strains 10791, 12266, and 20299. Because the phenotypes of the S. cerevisiae strains used in this example do not include tryptophan auxotrophy, the transformants were not expected to maintain the pESCTrp plasmids.

### Example 2

### Overexpression Of The GFA1 Gene in S. cerevisiae Strains and Accumulation Of Glucosamine and/or N-Acetylglucosamine in the Fermentation Broth

Induction of the GFA1 Gene

S. cerevisiae strains carrying the pESCHis plasmid with or without the GFA1 insert were grown in 5 mL SC-His containing 2% glucose overnight at 30° C. with shaking. One mL from each culture was transferred to 5 mL of SC-His medium containing 1% raffinose and 1% glucose and the incubation was continued for 10 h. The medium of the strains with single gene deletions also contained 0.2 mg/mL geneticin. The OD₆₀₀ of each culture was determined and the amount of culture necessary to obtain an OD₆₀₀ of 0.16 to 0.4 in 100 mL of SC-His containing 1% galactose and 1% raffinose (induction medium) was calculated. The calculated volume of cells was centrifuged at 1500×g for 10 min at 4° C. and the pellet was resuspended in 100 mL induction medium. Each construct was grown at 30° C. with shaking at 250 rpm from 0 to 90 h.

Determination of Glucosamine and N-Acetylglucosamine Formation

At 0, 13, 24, 48, 66 and 90 hours (h), aliquots of fermentation broth were removed, the OD₆₀₀ was measured, and the aliquots were centrifuged to remove the cells. The supernatants were frozen at −80° C. The cell pellet fractions from the aliquots harvested at 66 and 90 h were also frozen at −80° C.

In general the growth rates of the haploid strains expressing the GFA1 gene were substantially lower than the diploid strains. For example, the OD₆₀₀ measurements at 13 h and 48 h after induction for the haploid strain BY4742 were 1.69 and 4.14, respectively, while those of the diploid strain BY4743 were 2.44 and 5.27, respectively.

Product formation was determined in the thawed samples using the methods described in the Materials and Methods Section above. To distinguish between glucosamine and N-acetylglucosamine using the Elson and Morgan method, some of the assays were carried out with and without the acetylation step. The results of the Elson and Morgan assays of the fermentation broth samples are summarized in Tables 1 (with acetylation step) and 2 (without acetylation step). To confirm product formation, fermentation broth samples from transformed constructs of BY4742, BY4743, 23800, and 16545 were also analyzed by HPLC. The results of these analyses, measuring N-acetylglucosamine production, are shown in Table 3.

Strain BY4743 carrying the pESCHis vector or the vector with the GFA1 insert was analyzed for N-acetylglucosamine and glucosamine formation using the HPLC methods described in the Materials and Methods Section above. FIGS. 1 and 2 show chromatograms of the fermentation broth samples withdrawn 90 h after induction, demonstrating that this strain produces and secretes glucosamine, in addition to N-acetylglucosamine, when the GFA1 gene is overexpressed. Multiple standards were analyzed before each sample set. Standards and blanks were measured before and after each injection to verify that retention times were not affected by other broth components, and that carryover between injections was minimized. The glucosamine retention time did not vary by more than 0.01 minutes during the experimental sample set. The concentration of glucosamine hydrochloride in the broth was calculated to be 0.7 μg/mL (3.2 μM).

In a separate experiment the eleven strains listed in Example 1, carrying the pESCHis vector or the vector with the GFA1 insert, were induced in 50 mL of medium and assayed as described above. Samples were withdrawn at 0, 4, 8 and 21 h. The results of analysis of fermentation broth samples from this experiment using the Elson and Morgan assays (including the acetylation step) are summarized in Table 4.

TABLE 1
Product Formation assayed using the Elson and
Morgan method including the acetylation step

| | | [Product]; mM | | | | |
|---|---|---|---|---|---|---|
| Strain | Plasmid | 13 h | 24 h | 38 h | 66 h | 90 h |
| BY4742 | GFA1pESCHis | 0.05 | 0.29 | 0.46 | 0.62 | 0.71 |
| BY4743 | GFA1pESCHis | 0.09 | 0.29 | 0.38 | 0.41 | 0.43 |
| 23800 | GFA1pESCHis | 0.14 | 0.45 | 0.60 | 0.65 | 0.70 |
| 20324 | GFA1pESCHis | 0.08 | 0.29 | 0.35 | 0.33 | 0.36 |
| 20299 | GFA1pESCHis | 0.08 | 0.26 | 0.34 | 0.38 | 0.41 |
| 16545 | GFA1pESCHis | 0.01 | 0.13 | 0.64 | 1.34 | 1.63 |
| 15893 | GFA1pESCHis | 0.03 | 0.30 | 0.49 | 0.58 | 0.62 |
| 14977 | GFA1pESCHis | 0.03 | 0.21 | 0.43 | 0.60 | 0.66 |
| BY4742 | pESCHis | 0.00 | 0.03 | 0.03 | 0.01 | 0.01 |
| BY4743 | pESCHis | 0.00 | 0.01 | 0.01 | 0.00 | 0.02 |

TABLE 2
Product Formation assayed using the Elson and
Morgan method excluding the acetylation step

| | | [Product]; mM | | | | |
|---|---|---|---|---|---|---|
| Strain | Plasmid | 13 h | 24 h | 38 h | 66 h | 90 h |
| BY4742 | GFA1pESCHis | 0.04 | 0.26 | 0.43 | 0.62 | 0.68 |
| BY4743 | GFA1pESCHis | 0.09 | 0.30 | 0.33 | 0.40 | 0.43 |
| 23800 | GFA1pESCHis | 0.15 | 0.49 | 0.58 | 0.64 | 0.66 |
| 20324 | GFA1pESCHis | 0.08 | 0.29 | 0.35 | 0.33 | 0.34 |
| 20299 | GFA1pESCHis | 0.09 | 0.26 | 0.36 | 0.38 | 0.39 |
| 16545 | GFA1pESCHis | 0.01 | 0.15 | 0.61 | 1.33 | 1.57 |
| 15893 | GFA1pESCHis | 0.03 | 0.28 | 0.48 | 0.60 | 0.62 |
| 14977 | GFA1pESCHis | 0.03 | 0.21 | 0.41 | 0.60 | 0.66 |
| BY4742 | pESCHis | 0.00 | 0.01 | 0.03 | 0.01 | 0.02 |
| BY4743 | pESCHis | 0.00 | 0.01 | 0.01 | 0.00 | 0.01 |

TABLE 3
N-Acetylglucosamine Formation determined by HPLC

[N-Ac-glucosamine]; mM

| | | 90 h | 90 h |
|---|---|---|---|
| Strain | Plasmid | RI | UV |
| BY4742 | GFA1pESCHis | 0.60 | 0.66 |
| BY4742 | pESCHis | | 0.03 |
| BY4743 | GFA1pESCHis | 0.43 | 0.40 |
| BY4743 | pESCHis | | 0.02 |
| 23800 | GFA1pESCHis | 0.57 | 0.61 |
| 16545 | GFA1pESCHis | 1.85 | 1.70 |

TABLE 4
Product Formation assayed using the Elson and Morgan method
including the acetylation step (all constructs)

[Product]; mM

| Strain | Plasmid | 8 h | 21 h |
|---|---|---|---|
| BY4742 | pESCHis | 0.00 | 0.01 |
| BY4743 | pESCHis | 0.00 | 0.01 |
| 23800 | pESCHis | 0.00 | 0.01 |
| 20324 | pESCHis | 0.00 | 0.01 |
| 20299 | pESCHis | 0.00 | 0.01 |
| 25635 | pESCHis | 0.00 | 0.01 |
| 16545 | pESCHis | 0.00 | 0.00 |
| 15893 | pESCHis | 0.00 | 0.01 |
| 14977 | pESCHis | 0.00 | 0.01 |
| 12266 | pESCHis | 0.00 | 0.01 |
| 10791 | pESCHis | 0.00 | 0.00 |
| BY4742 | GFA1pESCHis | 0.042 | 0.25 |
| BY4743 | GFA1pESCHis | 0.058 | 0.36 |
| 23800 | GFA1pESCHis | 0.07 | 0.51 |
| 20324 | GFA1pESCHis | 0.03 | 0.23 |
| 20299 | GFA1pESCHis | 0.05 | 0.32 |
| 25635 | GFA1pESCHis | 0.03 | 0.29 |
| 16545 | GFA1pESCHis | 0.02 | 0.21 |
| 15893 | GFA1pESCHis | 0.00 | 0.35 |
| 14977 | GFA1pESCHis | 0.02 | 0.28 |
| 12266 | GFA1pESCHis | 0.03 | 0.30 |
| 10791 | GFA1pESCHis | 0.00 | 0.07 |

### Example 3

### Overexpression Of The GFA1 Gene and Accumulation of Glucosamine and/or N-Acetylglucosamine in the Fermentation Broth Using S. cerevisae Constructs Carrying Multiple Copies of the Gene

Induction of the GFA1 Gene

S. cerevisiae strains transformed using the 3 plasmids (pESCHis, pESCLeu, and pESCtrp) with or without 2 GFA1 inserts were grown in 5 mL SC-His containing 2% glucose overnight at 30° C. with shaking. One mL from each culture was transferred to 5 mL of SC-His-Trp-Leu medium containing 1% raffinose and 1% glucose and the incubation was continued for 9 h. The medium of the strains with single gene deletions also contained 0.2 mg/mL geneticin. The OD₆₀₀ of each culture was determined and the amount of culture necessary to obtain an OD₆₀₀ of 0.2 to 0.4 in 50 mL of SC-His-Trp-Leu containing 1% galactose and 1% raffinose (induction medium) was calculated. The calculated volume of cells was centrifuged at 1500×g for 10 min at 4° C. and the pellet was resuspended in 50 mL induction medium. Each construct was grown at 30° C. with shaking at 250 rpm from 0 to 36 h.

Determination of Glucosamine and/or N-Acetylglucosamine Formation

At 0, 11.5, 16, 21, and 35 hours, aliquots of fermentation broth were removed, the OD₆₀₀ was measured, and then the aliquots were centrifuged to remove the cells and the supernatants were frozen at −80° C.

Like the strains transformed with one copy of the GFA1 gene, the haploid strains transformed with multiple copies grew more slowly than the diploid strains. For example, the OD₆₀₀ measurements at 16 and 35 h after induction for the haploid strain BY4742 were 0.867 and 3.79, respectively, while those for the diploid strain BY4743 were 0.89 and 4.64, respectively.

Product formation was determined in the thawed samples using the methods described in the Materials and Methods section above. To distinguish between the two possible products, glucosamine and N-acetylglucoasmine, some of the Elson-Morgan assays were carried out with and without the acetylation step. In addition, selected samples were also analyzed by HPLC.

The results of the Morgan-Elson assays of the fermentation broth samples are summarized in Tables 5 (with acetylation step) and 6 (with and without acetylation reaction). The results of fermentation broth samples from transformed constructs of BY4742, BY4743, 23800, 25635, and 14977 analyzed for N-acetylglucosamine formation by HPLC are shown in Table 7. Little or no product could be detected in the strains carrying only the pESC vectors.

Cell extracts were prepared from frozen cell pellet samples harvested 35 h after induction. Cells from 10 mL of culture were suspended in 0.2 M HCl at ratios of 2:1 to 4:1 acid to cells and transferred to 1.7 mL tubes with screw caps and O-rings containing 1.4 g glass beads (0.5 mm). The cells were disrupted at 4° C. using a Mini-BeadBeater for 1 min at the homogenize setting and then were cooled in an ice-water bath for 1 min. The process was repeated twice. The tubes were centrifuged for 15 min at 21,000×g at 4 C and the supernatants were removed. Product formation was determined in the supernatant fractions (cell extracts) as described in the Materials and Methods section above using 0.02 mL per Elson and Morgan assay (see Table 8).

TABLE 5
Product formation in fermentation broth samples assayed by
the Elson and Morgan method (with the acetylation step)

| | | [Product]; mM | | | | |
|---|---|---|---|---|---|---|
| Strain | Plasmid* | 0 h | 11.5 h | 16 h | 21 h | 35 h |
| BY4742 | pESC | 0.00 | 0.00 | 0.00 | 0.00 | 0.00 |
| BY4743 | pESC | 0.00 | 0.00 | 0.00 | 0.00 | 0.00 |
| 16545 | pESC | 0.00 | 0.00 | 0.00 | 0.00 | 0.00 |
| 23800 | pESC | 0.00 | 0.00 | 0.00 | 0.00 | 0.00 |
| 14977 | pESC | 0.00 | 0.00 | 0.00 | 0.00 | 0.00 |
| 25635 | pESC | 0.00 | 0.00 | 0.00 | 0.00 | 0.00 |
| 15893 | pESC | 0.00 | 0.00 | 0.00 | 0.00 | 0.00 |
| 20324 | pESC | 0.00 | 0.00 | 0.00 | 0.00 | 0.00 |
| BY4742 | GFA1pESC | 0.00 | 0.00 | 0.04 | 0.15 | 0.61 |
| BY4743 | GFA1pESC | 0.00 | 0.06 | 0.20 | 0.43 | 0.92 |
| 16545 | GFA1pESC | 0.00 | 0.00 | 0.01 | 0.05 | 0.35 |
| 23800 | GFA1pESC | 0.00 | 0.02 | 0.15 | 0.35 | 0.70 |
| 14977 | GFA1pESC | 0.00 | 0.00 | 0.06 | 0.12 | 0.53 |
| 25635 | GFA1pESC | 0.00 | 0.03 | 0.14 | 0.24 | 0.57 |
| 15893 | GFA1pESC | 0.00 | 0.00 | 0.05 | 0.10 | 0.43 |
| 20324 | GFA1pESC | 0.00 | 0.03 | 0.13 | 0.22 | 0.67 |

*pESC and GFA1pESC denote the multiple plasmid transformation

TABLE 6
Product formation in fermentation broth samples harvested
at 35 h and assayed by the Elson and Morgan method
with and without the acetylation step

[Product]; mM

| Strain | Plasmid | +Acetylation Step | −Acetylation Step |
|---|---|---|---|
| BY4742 | GFA1pESC | 0.66 | 0.65 |
| BY4743 | GFA1pESC | 0.98 | 0.92 |
| 16545 | GFA1pESC | 0.39 | 0.38 |
| 23800 | GFA1pESC | 0.87 | 0.75 |
| 14977 | GFA1pESC | 0.62 | 0.56 |
| 25635 | GFA1pESC | 0.62 | 0.61 |
| 15893 | GFA1pESC | 0.47 | 0.44 |
| 20324 | GFA1pESC | 0.73 | 0.70 |

*GFA1pESC denotes the multiple plasmid transformation

TABLE 7
N-Acetyglucosamine formation assayed by HPLC

[N-Ac-glucosamine]; mM

| | | 35 h | 35 h |
|---|---|---|---|
| Strain | Plasmid | RI | UV |
| BY4742 | pESC | 0.00 | 0.02 |
| BY4743 | pESC | 0.00 | 0.02 |
| 23800 | pESC | 0.00 | 0.02 |
| 25635 | pESC | 0.00 | 0.01 |
| 14977 | pESC | 0.00 | 0.02 |
| BY4742 | GFA1pESC | 0.71 | 0.74 |
| BY4743 | GFA1pESC | 1.06 | 1.10 |
| 23800 | GFA1pESC | 0.89 | 0.94 |
| 25635 | GFA1pESC | 0.70 | 0.75 |
| 14977 | GFA1pESC | 0.66 | 0.69 |

*pESC and GFA1pESC denote the multiple plasmid transformation

TABLE 8
Product formation in cell extract samples harvested
at 35 h and assayed by the Elson and Morgan method
with and without the acetylation step

[Product]; mM

| Strain | Vector | +Acetylation Step | −Acetylation Step |
|---|---|---|---|
| BY4742 | GFA1pESC | 1.75 | 1.75 |
| BY4743 | GFA1pESC | 0.75 | 0.75 |
| 23800 | GFA1pESC | 1.35 | 1.35 |
| 20324 | GFA1pESC | 1.50 | 1.50 |
| 25635 | GFA1pESC | 0.50 | 0.50 |
| 14977 | GFA1pESC | 2.10 | 2.40 |
| 15893 | GFA1pESC | 2.40 | 2.85 |
| BY4743 | pESC | 0.00 | 0.00 |
| 23800 | pESC | −0.15 | 0.00 |
| 14977 | pESC | 0.15 | 0.15 |

### Example 4

### Growth Of S. cerevisiae Strains in the Presence of Glucosamine

S. cerevisiae strains BY4742 and BY4743 transformed with pESCHis carrying the GFA1 insert were grown in 5 mL SC-His medium containing 2% glucose overnight at 30° C. with shaking. An aliquot (0.5 mL) from each culture was transferred to 6 flasks containing 50 mL of SC-His medium containing 2% glucose. Glucosamine at the following concentrations was added to the flasks: 0, 2, 6, 10, 15, 20 mg/mL for each strain. The incubation was continued for 28 h at 30° C. with shaking. At 3, 16 and 28 h samples were withdrawn and the optical density at 600 nm was measured. These measurements are shown in Table 9.

TABLE 9
Effect of Glucosamine on the Growth of Two S. cerevisiae strains

| | | [glucosamine] | OD600 | OD600 | OD600 |
|---|---|---|---|---|---|
| Strain | vector | mg/mL | 3 h | 16 h | 28 h |
| BY4742 | GFA1pESCHis | 0.0 | 0.69 | 3.02 | 3.27 |
| BY4742 | GFA1pESCHis | 2.0 | 0.71 | 3.06 | 3.32 |
| BY4742 | GFA1pESCHis | 6.0 | 0.66 | 2.98 | 3.27 |
| BY4742 | GFA1pESCHis | 10.0 | 0.64 | 2.87 | 3.06 |
| BY4742 | GFA1pESCHis | 15.0 | 0.60 | 2.92 | 3.22 |
| BY4742 | GFA1pESCHis | 20.0 | 0.57 | 2.84 | 3.02 |
| BY4743 | GFA1pESCHis | 0.0 | 0.93 | 4.52 | 4.58 |
| BY4743 | GFA1pESCHis | 2.0 | 0.74 | 4.12 | 4.69 |
| BY4743 | GFA1pESCHis | 6.0 | 0.76 | 3.99 | 5.02 |
| BY4743 | GFA1pESCHis | 10.0 | 0.66 | 4.18 | 4.36 |
| BY4743 | GFA1pESCHis | 15.0 | 0.68 | 4.00 | 4.14 |
| BY4743 | GFA1pESCHis | 20.0 | 0.38 | 3.78 | 3.98 |

### Example 5

### Improvement of GFA1 (Glucosamine-6-Phosphate Synthase/Glutamine Fructose-6-Phosphate Amidotransferase) Activity Through Site-Specific Mutagenesis

Site-Specific Mutagenesis:

Site-specific mutagenesis is performed using the QuikChange Multi Site-Directed Mutagenesis kit (Stratagene; La Jolla, Calif.). The 5′-phosphorylated oligonucleotide primers are designed, based on guidelines provided by the manufacturer, to introduce the following specific amino acid changes:

I4T (ATC → ACC):
5′-CCAGAATGTGTGGTACCTTTGGTTACTGC-3′
D45T (GAC → ACC):
5′-GCTATCGATGGTACCGAAGCTGATTCTAC-3′
D353C (GAC → TGC):
5′-GAAGGGCCCTTACTGCCATTTTATGCAAAAGG-3′
I375T (ATC → ACC):
5′-CAATACTATGAGAGGTAGAACCGACTATGAAAA-3′
K553P (AAG → CCG):
5′-GCAGGTATTACCGCTGGAACCAAGAATAAAAAAGC-3′
L573P (TTG → CCG):
5′-GGATCAAAAATCTCTACCGTTATTGGGCAGAGGTTACC-3′
G576S (GGT → AGT):
5′-CTCTATTGTTATTGAGTAGAGGTTACCAATTTG-3′
L573P(TTG → CCG), G576S(GGT → AGT):
5′-GGATCAAAAATCTCTACCGTTATTGAGTAGAGGTTACC-3′

Note: Substituted amino acids are marked in italics; specific nucleotide substitutions are underlined. All primers are purified by polyacrylamide gel electrophoresis (PAGE). An additional L573P, G576S double mutant primer is described due to overlap of corresponding single mutation primers.

Saccharomyces cerevisiae GFA1 is cloned into the BamHI, XhoI site of pESC-His such that the orientation of the GFA1 gene is pGal1→BamHI→GFA1→XhoI (See EXAMPLE 1—Genetic construct designated GFA1pESCHis). The resulting plasmid is used as the template for multiple site-specific mutagenesis. All 8 mutagenic primers (50 ng of each) are added to a single reaction and the mutagenic reaction is performed according to the manufacturer's directions, based on the 8.8 kbp template. The mutagenesis reaction is allowed to proceed using the following cycling parameters:
1. 95° C. for 1 minute
2. 95° C. for 1 minute
3. 55° C. for 1 minute
4. 65° C. for 18 minutes
5. Repeat steps 2-4 29 times

E. coli XL-10 Gold (Stratagene; La Jolla, Calif.), transformed with the completed mutagenesis mix is allowed to recover for 1 hour at 37° C. with shaking (200-250 rpm). The recovered transformants are collected by centrifugation at 21,000×g for 30 seconds, transferred into 10 ml fresh LB+100 μg/ml ampicillin (Sigma-Aldrich; St. Louis, Mo.) and allowed to grow 16 hrs at 37° C. with shaking (200-250 rpm). Alternatively, recovered transformants are plated on LB+100 μg/ml ampicillin (Sigma-Aldrich; St. Louis, Mo.) and individual colonies are inoculated into LB broth+100 μg/ml ampicillin (Sigma-Aldrich; St. Louis, Mo.) and allowed to grow 16 hours at 37° C. with shaking (200-250 rpm). In both cases, plasmid DNA is recovered using the QIAQuick plasmid mini-prep kit.

Random Mutagenesis:

The recovered plasmid DNA is used as template for error prone PCR using the Diversify P CR Random Mutagenesis Kit (BD Biosciences Clontech; Palo Alto, Calif.). Reaction conditions are varied according to the manufacturer's directions until an error rate of 4-6 mutations per gene is achieved (typically corresponding to reaction conditions 1-3 (Diversify PCR Random Mutagenesis Kit User Manual)). Primers for random mutagenesis are:

| | |
|---|---|
| 1. | 5′-GAAAAAACCCCGGATCCAGAATGTG-3′ |
| 2. | 5′-GCGGTACCAAGCTTACTCGAGTTATTC-3′ |

The mutagenesis reaction is allowed to proceed using the following thermocycling parameters:
1. 94° C. for 30 seconds
2. 94° C. for 30 seconds
3. 68° C. for 2 minutes
4. Repeat steps 2-3 24 times

The PCR reaction is purified using the QIAquick PCR Purification Kit (Qiagen Inc.; Valencia, Calif.) and eluted in 50 μl EB buffer. The purified mutagenesis reaction is transformed into E. coli XL-10 Gold. Transformed cells are allowed to recover for 1 hour at 37° C. with shaking.

The recovered transformants are collected by centrifugation at 21,000×g for 30 seconds, transferred into 10 ml fresh LB+100 μg/ml ampicillin (Sigma-Aldrich; St. Louis, Mo.) and allowed to grow 16 hrs at 37° C. with shaking (200-250 rpm). Alternatively, recovered transformants are plated on LB+100 μg/ml ampicillin and individual colonies are inoculated into LB broth+ampicillin and allowed to grow 16 hours at 37° C. with shaking (200-250 rpm). In both cases, plasmid DNA is then recovered from the liquid cultures using the QIAquick Spin Miniprep Kit (Qiagen Inc.; Valencia, Calif.).

Activity Screening:

Purified plasmid is transformed into S. cerevisiae 24954 using the S.c. EasyComp Transformation Kit (Invitrogen; Carlsbad, Calif.). Individual S. cerevisiae transformants are transferred as stabs onto SC-His agar plates that have been freshly plated with a lawn of the S. cerevisiae glucosamine auxotrophic strain XW270-2D (MATα, gcn1-1, lys2-2; ATCC 52529). Successful mutants are chosen based on the large zone of growth surrounding each stab. Mutant clones are recovered and purified for the 24954 strain carrying the mutant GFA1 by growth on SC-His+200 μg/ml geneticin. Plasmid DNA carrying mutant GFA1 is purified from S. cerevisiae 24954 using the Zymoprep Yeast Plasmid Miniprep (Zymo Research; Orange, Calif.) kit and is transformed into E. coli XL-10 Gold and plated on LB+100 μg/ml ampicillin. Individual colonies are inoculated into LB+100 μg/ml ampicillin and grown 16 hours at 37° C. with shaking (200-250 rpm). Plasmid DNA is purified using the QIAquick Spin Miniprep Kit and the GFA1 sequences are determined using the Dideoxy (Sanger) method (1977) of DNA sequencing. Mutant GFA1 genes are subjected to further rounds of site-specific and random mutagenesis to generate further improvements in activity.

Alternatively, individual S. cerevisiae transformants are transferred as stabs onto plates that have been freshly plated with a lawn of an E. coli strain carrying an inactivated glmS gene (generated, for example, using the technique of Datsenko and Wanner). Datsenko K A and Wanner BL (2000). One-Step inactivation of chromosomal genes in Escherichia coli K-12 using polymerase chain reaction products. Proc. Natl. Acad. Sci. USA. 97:6640-6645.

Successful mutants are chosen based on the large zone of growth surrounding each stab. Mutant clones are recovered and purified for the 24954 strain carrying the mutant GFA1 by growth on LB+100 μg/ml ampicillin. Plasmid DNA carrying mutant GFA1 is purified from S. cerevisiae 24954 using the Zymoprep Yeast Plasmid Miniprep (Zymo Research; Orange, Calif.) kit and is transformed into E. coli XL-10 Gold and plated on LB+100 μg/ml ampicillin. Individual colonies are inoculated into LB+100 μg/ml ampicillin and grown 16 hours at 37° C. with shaking (200-250 rpm). Plasmid DNA is purified using the QIAquick Spin Miniprep Kit and the GFA1 sequences are determined using the Dideoxy (Sanger) method of DNA sequencing. Mutant GFA1 genes are subjected to further rounds of site-specific and random mutagenesis to generate further improvements in activity.

### Example 6

### Disruption of UAP1 (UDP-N-acetylglucosamine pyrophosphorylase) in a Homozygous Diploid Mutant of the PGM2 Gene

The following example describes the procedure for disruption of one copy of the UAP1 gene in a homozygous diploid mutant of the PGM2 gene. The resulting mutant is expected to be a particularly suitable host for overexpression of the GFA1 gene and high level production of N-acetylglucosamine.

S. cerevisiae gene UAP1 (UDP-N-acetylglucosamine pyrophosphorylase) is disrupted by the insertion of the nourseothricin drug resistance cassette directly into the UAP1 chromosomal gene sequence. The UAP1 gene disruption cassette is constructed by PCR-mediated amplification of plasmid pAG25 (European Saccharomyces Cerevisiae Archive for Functional Analysis Accession # P30104, Frankfurt, Germany). Plasmid pAG25 is constructed by replacing the kanamycin resistance cassette open reading frame from the kanMX4 disruption cassette (Wach et al., 1994) with the nat1 gene (nourseothricin N-acetyltransferase) from Streptomyces noursei to generate natMX4 (Goldstein and McCusker, 1999). Wach et al. (1994) Yeast 10: 1793-1808; Goldstein A L and McCusker J H. (1999) Yeast 15: 1541-1553.

PCR Amplification of the UAP1 Gene Disruption Cassette from natMX4 Template:

50 uL reactions are prepared using the PCR optimization kit OptiPrime PCR Optimization kit (Stratagene; Cedar Creek, Tex.) according to the supplier's directions. Amplification of the cassettes follow the protocol (1) 94° C. for 1 minute (2) 94° C. for 1 minute (3) 55° C. for 1 minute (4) 72° C. for 3 minute (5) repeat steps 2-4 30 times (6) 72° C. for 20 minutes.

Oligonucleotide primers for the amplification reaction are as follows:

Primer (1)
5′ - CAAAGAAACGCAGATATAAAGGAGAACACCAGATCGAACTAGCT
TCGTACGCTGCAGGTC - 3′
Primer (2)
5′ - TACATGTTAAAAGTGCTTCATAAGTAGCTCAAACAGCCGCATAG
GCCACTAGTGGATC - 3′

Underlined sequences identify homology to the natMX4 template. Sequences in bold are homologous to S. cerevisiae sequence surrounding the UAP1 gene.

5 uL aliquots of the PCR reactions are applied to a 1% agarose gel and PCR products are visualized by staining with ethidium bromide. The PCR reaction condition judged to produce the highest yield of the desired product is used to produce approximately 2 ug of product. The PCR product is purified by gel purification (QIAquick Gel Extraction Kit; Qiagen Inc., Valencia Calif.) and 1-2 μg of gel-purified product is used to transform S. cerevisiae deletion strain 36545 (PGM2 homozygous diploid; Invitrogen Corp.; Carlsbad, Calif.) using the S. cerevisiae EasyComp transformation kit (Invitrogen Corp.; Carlsbad, Calif.).

The transformed cells are allowed to grow 2-4 hours in YPAD at 30° C. on an orbital shaker set to 200 rpm. Cultures transformed with the natMX3 disruption cassette are plated onto the selective media YPAD+100 μg/ml nourseothicin (clonNAT; Werner BioAgents; Jena-Cospeda, Germany)+200 μg/ml geneticin (Sigma Aldrich Inc.; St. Louis, Mo.).

Genomic DNA is purified (Ausubel et al., 1995) from cultures grown from isolated colonies in YPAD+100 μg/ml clonNAT+200 μg/ml geneticin. Ausubel et al. (1995) Current Protocols in Molecular Biology. Wiley Interscience. New York. Ausubel Integration of the drug resistance cassette is verified by PCR following the protocol (1) 94° C. for 1 minute (2) 94° C. for 91 minute (3) 55° C. for 1 minute (4) 72° C. for 90 seconds (5) repeat steps 2-4 30 times (6) 72° C. for 7 minutes. Confirmation of gene disruption is through the generation of a 0.9-1.0 Kbp PCR product.

Oligonucleotide primers for the amplification reaction to confirm cassette integration are as follows:

Primer (3)
5′ -CACTAACCAGCAGGCTAACA- 3′
Primer (4)
5′ -TTCGCCTCGACATCATCT- 3′

Note: Oligonucleotide primer (3) is homologous to the region of the S. cerevisiae genome downstream of the UAP1 gene. Oligonucleotide primer (4) is homologous to the gene disruption cassette (downstream of nat1 Open Reading Frame). Insertion of the gene disruption cassette (natMX4) into the UAP1 gene (in the correct orientation) will result in the correct PCR amplification product being generated.

Similarly, this protocol can be used to disrupt the S. cerevisiae UAP1 gene using the hygromycin B phosphotransferase (hph) gene from Klebsiella pneumoniae as the dominant selectable marker. In this case the drug resistance cassette is carried on the plasmid pAG32 (European Saccharomyces Cerevisiae Archive for Functional Analysis Accession # P30104, Frankfurt, Germany). Plasmid pAG32 was constructed by replacing the kanamycin resistance cassette open reading frame from the kanMX4 disruption cassette (Wach et al., 1994) with the hph gene (hygromycin B phosphotransferase) from Klebsiella pneumoniae to generate hphMX4 (Goldstein and McCusker, 1999).

Generation of the UAP1 gene disruption cassette is performed as above with the exception that plasmid pAG32 replaces pAG25 as template in the PCR-mediated amplification of the UAP1 disruption cassette. S. cerevisiae strain 36545 containing the UAP1 gene disrupted by the hphMX4 cassette can be selected for by replacing 100 μg/ml nourseothicin (clonNAT; Werner BioAgents; Jena-Cospeda, Germany) with 300 μg/ml hygromycin B (Sigma-Aldrich, St. Loius, Mo.) as the selective agent. All other steps are performed as described above.

Similarly, this protocol can be used to disrupt the S. cerevisiae UAP1 gene using the phosphinothricin N-acetyltransferase (pat) gene from Streptomyces viridochromogenes Tu94 as the dominant selectable marker. In this case the drug resistance cassette is carried on the plasmid pAG29 (European Saccharomyces Cerevisiae Archive for Functional Analysis Accession # P30105, Frankfurt, Germany). Plasmid pAG29 was constructed by replacing the kanamycin resistance cassette open reading frame from the kanMX4 disruption cassette (Wach et al., 1994) with the pat gene (phosphinothricin N-acetyltransferase) from Streptomyces viridochromogenes Tu94 to generate patMX4 (Goldstein and McCusker, 1999).

Generation of the UAP1 gene disruption cassette is performed as described above with the exception that plasmid pAG29 replaces pAG25 as template in the PCR-mediated amplification of the UAP1 disruption cassette. S. cerevisiae strain 36545 containing the UAP1 gene disrupted by the patMX4 cassette can be selected for by growing the transformed cells for 2-4 hours in SDP at 30° C. on an orbital shaker set to 200 rpm and replacing YPAD+100 μg/ml nourseothicin (clonNAT; Werner BioAgents; Jena-Cospeda, Germany)+200 μg/ml geneticin (Sigma Aldrich; St. Louis, Mo.) with SDP+600-1000 μg/ml gluphosinate (Sigma-Aldrich, St. Louis, Mo.)+200 μg/ml geneticin (Sigma Aldrich; St. Louis, Mo.). All other steps are performed as described above.

Protocols using the kan MX4, natMX4, hphMX4 and patMX4 can be modified to use the derivative kanMX3, natMX3, hphMX3 and patMX3 disruption cassettes. The kanMX3, natMX3, hphMX3 and patMX3 disruption cassettes have been modified to include include 466 bp direct repeats that flank the gene disruption cassette. This facilitates homologous recombination and loss of the marker cassette after disruption of the gene of interest. Marker loss results in loss of all sequence contained within the direct repeats as well as one copy of the repeat. Marker loss can be selected for by constructing fusions between the kan^{r}, nat1, hph or pat gene and the GAL1 gene of Candida albicans. Constituitive expression of GAL1 in the presence of 2-deoxygalactose is toxic to S. cerevisiae. Consequently, loss of the kan^{r}-GAL1, nat1-GAL1, hph-GAL1 or pat-GAL1 gene fusions will confer resistance to 2-deoxygalactose.

Alternatively, elimination of the marker cassettes can be accomplished by generation of PCR-mediated amplification of DNA sequences corresponding to either side of the disruption cassette's site of insertion. These flanking DNA sequences (each product 0.5 to 1.0 Kbp in length) are fused in a second round of PCR using overlap extension PCR (the 2 original PCR products must have short regions of homology (approximately 15-30 bp) to each other on the ends nearest to the gene disruption). The final product (1-2 μg) is transformed into the gene-disrupted host and transformants are screened (or selected if using GAL1 fusions and growth on 2-deoxygalactose) for loss of the dominant selectable marker being excised.

Other S. cerevisiae host genes can be disrupted using a modification of the above protocol. DNA sequences of oligonucleotide primer 1) and oligonucleotide primer 2) are modified such that the DNA sequences homologous to the UAP1 region of the S. cerevisiae genome (shown in bold) are made homologous to the S. cerevisiae host gene (or sequences surrounding the gene) that is to be disrupted. Selection for transformed cells carrying the disrupted gene are performed as above. Confirmation of gene disruption is performed as above with the exception that the sequences of oligonucleotide primers 3) and 4) are modified as appropriate.

If desired, host strains with multiple gene disruptions can be constructed by disrupting, first one desired gene with a dominant selectable disruption marker (for example S. cerevisiae deletion strain 36545 disrupted in the UAP1 gene by natMX4) as described above. The resulting strain (disrupted in PGM2 and UAP1 and capable of growth on YPAD+100 μg/ml nourseothicin (clonNAT; Werner BioAgents; Jena-Cospeda, Germany)+200 μg/ml geneticin (Sigma Aldrich; St. Louis, Mo.)) can then be disrupted in a separate gene (for example PFK26) using a third selectable marker (for example hphMX4) and selected for by growth on YPAD+100 μg/ml nourseothicin (clonNAT; Werner BioAgents; Jena-Cospeda, Germany)+200 μg/ml geneticin (Sigma Aldrich; St. Louis, Mo.)+300 μg/ml hygromycin B (Sigma-Aldrich, St. Loius, Mo.). Additional selectable markers can be incorporated as appropriate.

Additionally, gene disruption cassettes that complement selected host strain auxotrophies (for example methionine auxotrophy due to loss of met15 gene function) can be used to disrupt selected host genes. Gene disruption is performed using the appropriate complementing gene in the disruption cassette (for example using the met15, lys2 or ura3 genes to disrupt a host gene(s) of interest). In this case, selection for host strains with the appropriate gene disruption is performed by growth of the gene-disrupted host on media lacking the desired metabolite (for example, media lacking methionine, lysine or uracil).

### Example 7

### Deacetylation of N-Acetylglucosamine

Test broth samples without biomass comprising aqueous solutions of N-acetylglucosamine, glucosamine, dextrose, sodium chloride, and sodium acetate were acidified with 35% hydrochloric acid to a final HCl concentration of 3%, 6.9% or 10.8% by weight. The acidified broths were heated to 95° C. or 118° C. Samples of the broths were tested after 0, 1, 2, and 4 hours using the chromatographic methods described in the Materials and Methods section above. Results of the hydrolysis experiments are summarized in Tables 10 and 11, below.

TABLE 10
N-acetylglucosamine and glucosamine concentrations
as functions of time and HCl concentrations during
N-acetylglucosamine hydrolysis at 95° C.

| | | Wt % | Wt % |
|---|---|---|---|
| Temperature/time | % HCl | N-acetylglucosamine | Glucosamine |
| After acid addition | 3.0 | 6.70 | <0.1 |
| 95° C., 1 hour | 3.0 | 0.57 | 5.94 |
| 95° C., 2 hours | 3.0 | 0.073 | 6.39 |
| 95° C., 4 hours | 3.0 | <0.02 | 6.36 |
| After acid addition | 6.9 | 6.40 | <0.1 |
| 95° C., 1 hour | 6.9 | <0.02 | 6.30 |
| 95° C., 2 hours | 6.9 | <0.02 | 6.31 |
| 95° C., 4 hours | 6.9 | <0.02 | 6.18 |
| After acid addition | 10.8 | 6.34 | 0.22 |
| 95° C., 1 hour | 10.8 | <0.02 | 6.18 |
| 95° C., 2 hours | 10.8 | <0.02 | 6.10 |
| 95° C., 4 hours | 10.8 | <0.02 | 5.89 |

TABLE 11
N-acetylglucosamine and glucosamine concentrations
as functions of time and HCl concentrations during
N-acetylglucosamine hydrolysis at 118° C.

| | | Wt % | Wt % |
|---|---|---|---|
| Temperature/time | % HCl | N-acetylglucosamine | Glucosamine |
| After acid addition | 3.0 | 6.70 | <0.1 |
| 118° C., 1 hour | 3.0 | <0.02 | 6.34 |
| 118° C., 2 hours | 3.0 | <0.02 | 6.22 |
| 118° C., 4 hours | 3.0 | <0.02 | 6.07 |
| After acid addition | 6.9 | 6.40 | <0.1 |
| 118° C., 1 hour | 6.9 | <0.02 | 6.17 |
| 118° C., 2 hours | 6.9 | <0.02 | 6.06 |
| 118° C., 4 hours | 6.9 | <0.02 | 5.57 |
| After acid addition | 10.8 | 6.34 | 0.22 |
| 118° C., 1 hour | 10.8 | <0.02 | 6.04 |
| 118° C., 2 hours | 10.8 | <0.02 | 5.90 |
| 118° C., 4 hours | 10.8 | <0.02 | 5.37 |

### Example 8

### Cloning of the STE3 Gene into S. cerevisiae Strains

The nucleic acid sequence of STE3 (coding for mating-type a-factor pheromone receptor), SEQ ID NO: 5, is obtained from the Stanford yeast genome database. The STE3 gene is cloned into the pESCUra vector singly behind the Gall promoter or behind both the Gal1 and Gal10 promoters. Primers for the synthesis of the gene with appropriate restriction sequences for the pESCUra vector 5‘of the gene’s ATG start codon and 3‘of each gene’s stop codon are designed for PCR amplification using S. cerevisiae genomic DNA as template.

Forward primer for STE3 with BamHI site:
5′- CGCGGATCCAGAATGTCATACAAGTCAGCAATAATAG - 3′
Reverse primer for STE3 with XhoI site:
5′- AAGCTCGAGTTAAGGGCCTGCAGTATTTT - 3′
Forward primer for STE3 with SpeI site:
5′- GGACTAGTATGTCATACAAGTCAGCAATAATAGG- 3′
Reverse primer for STE3 with SacI site:
5′- AATGAGCTCTTAAGGGCCTGCAGTATTTTCT - 3′

Note: Italics indicate the restriction sites while bold lettering indicates the start and stop codons.

Construction of STE3pESCUra

The STE3 gene is amplified by PCR using the primers with BamHI and XhoI restriction sites. The thermocycler program used includes a hot start at 96° C. for 5 min; 10 repetitions of the following steps: 94° C. for 30 sec, 60-72° C. for 1 min, 45 sec (gradient thermocycler), and 72° C. for 1 min, 30 sec; 15 repetitions of the following steps: 94° C. for 30 sec, 60-72° C. for 1 min, 45 sec and 72° C. for 1 min, 30 sec increasing 5 sec each cycle; 10 repetitions of the following steps: 94° C. for 30 sec, 60-72° C. for 1 min, 45 sec and 72° C. for 2 min, 45 sec. After the 35 cycles the sample is incubated at 72° C. for 7 min and then stored at 4° C. The PCR product is purified from a 1% TAE-agarose gel (QIAQuick Gel Purification kit) and restriction digestion of both the PCR product and the pESCUra vector with BamHI and XhoI, the ligation is carried out using the Rapid DNA Ligation Kit (Roche). The ligation mixture is desalted and then transformed into E. coli DH10B ElectroMAX cells using the BioRad recommended procedure for transformation of E. coli cells with 0.2 cm micro-electroporation cuvettes. After recovery in SOC medium the transformation mixture is plated on LB plates containing ampicillin at 100 μg/mL. Plasmid DNA is isolated from liquid cultures [5 mL 2xYT medium+ampicillin (100 μg/mL) grown overnight at 37° C.] of colonies picked from the LB+ampicillin (100 μg/mL) plates and purified. The plasmids are then screened by restriction digestion and the sequences are verified by dideoxynucleotide chain-termination DNA sequencing.

Construction of 2(STE3)pESCUra

The STE3 gene is amplified by PCR using the primers with SpeI and SacI restriction sites. The PCR product is purified from a 1% TAE-agarose gel (QIAQuick Gel Purification Kit) and the sequence is verified by dideoxynucleotide chain-termination DNA sequencing. The STE3pESCUra plasmid and the PCR product are digested with SpeI and SacI. The plasmid is purified from a 1% TAE-agarose gel while the restriction digest mixture of the PCR product is purified using a QIAQuick PCR Clean-up kit. Ligations and transformations into E. coli DH10B cells are carried out as described above. Plasmid DNA is purified from ampicillin resistant cells and screened by restriction digestion. Plasmids carrying two copies of the STE3 gene are chosen for transformation into the S. cerevisiae strains.

Competent cells of the S. cerevisiae strains listed below are prepared using an S.c. EasyComp™ Transformation Kit (Invitrogen Corp). Aliquots (50 μL) are frozen at −80° C. and thawed just prior to use.

S. cerevisiae Strains

| | |
|---|---|
| BY4741 | haploid parental strain (MATa his3-Δ1, leu2-Δ0, met15-Δ0, |
| | ura3-Δ0) |
| 5645 | STE2 (YFL026W) deletion (alpha-factor pheromone receptor) |
| | (MATa haploid) |

Transformations of the STE3pESCUra vector construct as well as parent vectors and GFA1pESC vectors described in Example 1 into S. cerevisiae competent cells are also carried out using the S.c. EasyComp Transformation Kit. The vectors GFA1pESCHis and either STE3pESCUra or pESCUra are transformed simultaneously into each haploid strain. A 100 μL aliquot from each transformation reaction is spread on SC-His-Ura plates (medium recipes from Stratagene pESC manual). The plate medium of the 5645 strain also contains 0.2 mg/mL geneticin. The plates are incubated for 2 days at 30° C. Colonies from each plate are used to inoculate 5 mL liquid cultures of SC-His-Ura medium. The cultures are incubated overnight at 30° C. and the cells are harvested by centrifugation, and plasmid DNA is isolated from the cells using a Zymoprep Yeast Plasmid Miniprep kit. After analysis of the isolated DNA by PCR, one isolate from each of the 4 haploid constructs that generated the predicted PCR products is chosen for expression studies. Production of glucosamine and N-acetylglucosamine is compared among the 4 haploid constructs.

Alternatively, multiple copies of STE3 and/or GFA1 can be expressed using pESCHis, pESCUra and pESCLeu. The GFA 1 gene chosen for expression can be either the wild-type copy or an improved copy as described in Example 5.

The GFA1 and STE3 gene(s) that have been transformed as described above are overexpressed in shake flask experiments as described in Examples 2 and 3. Glucosamine and N-acetylglucosamine are measured in the fermentation broth using the methods described in the Materials and Methods section above. Glucosamine is purified from the fermentation broth as described in Example 7.

### Example 9

### Disruption of One Copy of the GNA1 Gene in a Homozygous Diploid Mutant of the PGM2 Gene

The following example describes the procedure for disruption of one copy of the GNA1 gene in a homozygous diploid mutant of the PGM2 gene. The resulting mutant is expected to be a particularly suitable host for overexpression of the GFA 1 gene and high level production of glucosamine.

Saccharomyces cerevisiae gene GNA1 (glucosamine-phosphate N-acetyltransferase) is disrupted by the insertion of the phosphinothricin drug resistance cassette directly into the GNA1 chromosomal gene sequence. The GNA1 gene disruption cassette is constructed by PCR-mediated amplification of plasmid pAG29 (European Saccharomyces Cerevisiae Archive for Functional Analysis Accession # P30105, Frankfurt, Germany). Plasmid pAG29 is constructed by replacing the kanamycin resistance cassette open reading frame from the kanMX4 disruption cassette (Wach A, Brachat A, Pohlmann R and Philippsen P. (1994). New heterologous modules for classical or PCR-based gene disruptions in Saccharomyces cerevisiae. Yeast. 10: 1793-1808.) with the pat gene (phosphinothricin N-acetyltransferase) from Streptomyces viridochromogenes Tu94 to generate patMX4 (Goldstein A L and McCusker J H. (1999). Three New Dominant Drug Resistance Cassettes for Gene Disruption in Saccharomyces cerevisiae. Yeast. 15: 1541-1553). PCR amplification of the GNA1 gene disruption cassette from patMX4 template is performed as follows:

50 uL reactions are prepared using the PCR optimization kit OptiPrime PCR Optimization kit (Stratagene; Cedar Creek, Tex.) according to the supplier's directions. Amplification of the cassettes follow the protocol (1) 94° C. for 1 minute (2) 94° C. for 1 minute (3) 55° C. for 1 minute (4) 72° C. for 3 minute (5) repeat steps 2-4 30 times (6) 72° C. for 20 minutes.

Oligonucleotide primers for the amplification reaction are as follows:

Primer (1)
5′ - GCTTACCCGATGGATTTTATATAAGGCGAATGGAAGAGGGAGCTT
CGTACGCTGCAGGTC - 3′
Primer (2)
5′ - AATGGAGATAAATGGTGAAGACCCTGCCAATAACCACCAGCCGCA
TAGGCCACTAGTGGATC - 3′

Underlined sequences identify homology to the patMX4 template. Sequences in bold are homologous to S. cerevisiae sequence surrounding the GNA1 gene.

5 uL aliquots of the PCR reactions are applied to a 1% agarose gel and PCR products are visualized by staining with Ethidium Bromide. The PCR reaction condition judged to produce the highest yield of the desired product is used to produce approximately 2 ug of product. The PCR product is purified by gel purification (QIAquick Gel Extraction Kit; Qiagen Inc., Valencia Calif.) and 1-2 μg of gel-purified product is used to transform S. cerevisiae deletion strain 36545 (PGM2 homozygous diploid; Invitrogen Corp.; Carlsbad, Calif.) using the S. cerevisiae EasyComp transformation kit (Invitrogen Corp.; Carlsbad, Calif.).

The transformed cells are allowed to grow 2-4 hours in SDP at 30° C. on an orbital shaker set to 200 rpm. Cultures transformed with the patMX3 disruption cassette are plated onto the selective media SDP+600-1000 μg/ml gluphosinate (Sigma Aldrich Inc.; St. Louis, Mo.)+200 μg/ml geneticin (Sigma Aldrich Inc.; St. Louis, Mo.). Genomic DNA is purified (Ausubel F M, Brent R, Kingston R E, Moore D D, Smith J A, Seidman J G and Struhl K. (eds) (1995). Current Protocols in Molecular Biology. Wiley Interscience. New York.) from cultures grown from isolated colonies in SDP+600-1000 μg/ml gluphosinate (Sigma Aldrich Inc.; St. Louis, Mo.)+200 μg/ml geneticin (Sigma Aldrich Inc.; St. Louis, Mo.). Integration of the drug resistance cassette is verified by PCR following the protocol (1) 94° C. for 1 minute (2) 94° C. for 91 minute (3) 55° C. for 1 minute (4) 72° C. for 90 seconds (5) repeat steps 2-4 30 times (6) 72° C. for 7 minutes. Confirmation of gene disruption is through the generation of a 0.7-0.8 Kbp PCR product.

Oligonucleotide primers for the amplification reaction to confirm cassette integration are as follows:

Primer (3)
5′ -CGCGGAGACTTCTCGCCAAT- 3′
Primer (4)
5′ -TTCGCCTCGACATCATCT- 3′

Note: Oligonucleotide primer (3) is homologous to the region of the S. cerevisiae genome downstream of the GNA1 gene. Oligonucleotide primer (4) is homologous to the gene disruption cassette (downstream of pat Open Reading Frame). Only by insertion of the gene disruption cassette (patMX4) into the GNA1 gene (in the correct orientation) will the correct PCR amplification product be generated.

Similarly, this protocol can be used to disrupt the S. cerevisiae GNA1 gene using the hygromycin B phosphotransferase (hph) gene from Klebsiella pneumoniae as the dominant selectable marker. In this case the drug resistance cassette is carried on the plasmid pAG32 (European Saccharomyces Cerevisiae Archive for Functional Analysis Accession # P30104, Frankfurt, Germany). Plasmid pAG32 was constructed by replacing the kanamycin resistance cassette open reading frame from the kanMX4 disruption cassette (Wach et al., 1994) with the hph gene (hygromycin B phosphotransferase) from Klebsiella pneumoniae to generate hphMX4 (Goldstein and McCusker, 1999).

Generation of the GNA1 gene disruption cassette is performed as above with the exception that plasmid pAG32 replaces pAG29 as template in the PCR-mediated amplification of the GNA1 disruption cassette. S. cerevisiae strain 36545 containing the GNA1 gene disrupted by the hphMX4 cassette can be selected for by growing the transformed cells for 2-4 hours in YPAD at 30° C. on an orbital shaker set to 200 rpm and replacing SDP+600-1000 μg/ml gluphosinate (Sigma Aldrich; St. Louis, Mo.)+200 μg/ml geneticin (Sigma Aldrich; St. Louis, Mo.) with YPAD+300 μg/ml hygromycin B (Sigma-Aldrich, St. Louis, Mo.)+200 μg/ml geneticin (Sigma Aldrich; St. Louis, Mo.). All other steps are performed as described above.

Similarly, this protocol can be used to disrupt the S. cerevisiae GNA1 gene using the nourseothricin N-acetyltransferase (nat1) gene from Streptomyces noursei as the dominant selectable marker. In this case the drug resistance cassette is carried on the plasmid pAG25 (European Saccharomyces Cerevisiae Archive for Functional Analysis Accession # P30104, Frankfurt, Germany). Plasmid pAG25 was constructed by replacing the kanamycin resistance cassette open reading frame from the kanMX4 disruption cassette (Wach et al., 1994) with the nat1 gene (nourseothricin N-acetyltransferase) from Streptomyces noursei to generate natMX4 (Goldstein and McCusker, 1999).

Generation of the GNA1 gene disruption cassette is performed as described above with the exception that plasmid pAG25 replaces pAG29 as template in the PCR-mediated amplification of the GNA1 disruption cassette. S. cerevisiae strain 36545 containing the GNA1 gene disrupted by the natMX4 cassette can be selected for by growing the transformed cells for 2-4 hours in YPAD at 30° C. on an orbital shaker set to 200 rpm and replacing SDP+600-1000 μg/ml gluphosinate (Sigma Aldrich; St. Louis, Mo.)+200 μg/ml geneticin (Sigma Aldrich; St. Louis, Mo.) with YPAD+100 μg/ml nourseothricin (clonNAT; Werner BioAgents; Jena-Cospeda, Germany)+200 μg/ml geneticin (Sigma Aldrich; St. Louis, Mo.). All other steps are performed as described above.

Protocols using the kan MX4, natMX4, hphMX4 and patMX4 can be modified to use the derivative kanMX3, natMX3, hphMX3 and patMX3 disruption cassettes. The kanMX3, natMX3, hphMX3 and patMX3 disruption cassettes have been modified to include 466 bp direct repeats that flank the gene disruption cassette. This facilitates homologous recombination and loss of the marker cassette after disruption of the gene of interest. Marker loss results in loss of all sequence contained within the direct repeats as well as one copy of the repeat. Marker loss can be selected for by constructing fusions between the kan^{r}, nat1, hph or pat1 gene and the GAL1 gene of Candida albicans. Constitutive expression of GAL1 in the presence of 2-deoxygalactose is toxic to S. cerevisiae. Consequently, loss of the kan^{r}-GAL1, nat1-GAL1, hph-GAL1 or pat1-GAL1 gene fusions will confer resistance to 2-deoxygalactose.

Alternatively, elimination of the marker cassettes can be accomplished by generation of PCR-mediated amplification of DNA sequences corresponding to either side of the disruption cassette's site of insertion. These flanking DNA sequences (each product 0.5 to 1.0 Kbp in length) are fused in a second round of PCR using overlap extension PCR (the 2 original PCR products must have short regions of homology (approximately 15-30 bp) to each other on the ends nearest to the gene disruption). The final product (1-2 μg) is transformed into the gene-disrupted host and transformants are screened (or selected if using GAL 1 fusions and growth on 2-deoxygalactose) for loss of the dominant selectable marker being excised.

Other S. cerevisiae host genes can be disrupted using a modification of the above protocol. DNA sequences of oligonucleotide primer 1) and oligonucleotide primer 2) are modified such that the DNA sequences homologous to the GNA1 region of the S. cerevisiae genome (shown in bold) are made homologous to the S. cerevisiae host gene (or sequences surrounding the gene) that is to be disrupted. Selection for transformed cells carrying the disrupted gene are performed as above. Confirmation of gene disruption is performed as above with the exception that the sequences of oligonucleotide primers 3) and 4) are modified as appropriate.

If desired, host strains with multiple gene disruptions can be constructed by disrupting, first one desired gene with a dominant selectable disruption marker (for example S. cerevisiae deletion strain 36545 disrupted in the GNA1 gene by patMX4) as described above. The resulting strain (disrupted in PGM2 and GNA1 and capable of growth on SDP+600-1000 μg/ml gluphosinate (Sigma Aldrich; St. Louis, Mo.)+200 μg/ml geneticin (Sigma Aldrich; St. Louis, Mo.)) can then be disrupted in a separate gene (for example PFK26) using a third selectable marker (for example hphMX4) and selected for by growth on SDP+600-1000 μg/ml gluphosinate (Sigma Aldrich; St. Louis, Mo.)+200 μg/ml geneticin (Sigma Aldrich; St. Louis, Mo.)+300 μg/ml hygromycin B (Sigma-Aldrich, St. Loius, Mo.). Additional selectable markers can be incorporated as appropriate.

Additionally, gene disruption cassettes that complement selected host strain auxotrophies (for example methionine auxotrophy due to loss of met15 gene function) can be used to disrupt selected host genes. Gene disruption is performed using the appropriate complementing gene in the disruption cassette (for example using the met15, lys2 or ura3 genes to disrupt a host gene(s) of interest). In this case, selection for host strains with the appropriate gene disruption is performed by growth of the gene-disrupted host on media lacking the desired metabolite (for example, media lacking methionine, lysine or uracil).

The GFA1 gene(s) described in Example 1 or 5 are cloned into the pESC vectors singly behind the Gal1 promoter or doubly behind both the Gal1 and Gal10 promoters as explained in Example 1. Transformations of the vector constructs into competent cells of the host strain are carried out as in Example 1.

The GFA1 gene(s) is overexpressed in shake flask experiments as described in Examples 2 and 3. Glucosamine and N-acetylglucosamine are measured in the fermentation broth using the methods described in the Materials and Methods Section above. Glucosamine is purified from the fermentation broth as described in Example 7.

### Example 10

### Cloning Of nagA Genes into Escherichia coli and Saccharomyces cerevisiae

The following example describes the cloning of nagA genes into Escherichia coli and Saccharomyces cerevisiae. The gene nagA codes for the enzyme N-acetylglucosamine-6-phosphate deacetylase.

Recombinant DNA techniques for PCR, purification of DNA, ligations and transformations were carried out according to established procedures (Sambrook and Russell, 2001, Molecular Cloning A Laboratory Manual, 3^{rd} Edition, Cold Spring Harbor Laboratory Press, Cold Spring Harbor, N.Y.). The sequence of the nagA gene from E. coli (coding for N-acetylglucosamine-6-phosphate deacetylase), SEQ ID NO: 3, was obtained from the NCBI nucleotide database (ACCESSION D90707; REGION: complement (1431.2579); VERSION D90707.1 GI:1651283).

The nagA gene from E. coli was cloned into the pET30(Xa/LIC) according to the manufacturer's protocol. Primers were designed with compatible overhangs for the pET 30 Xa/LIC vector (Novagen, Madison, Wis.). The pET vector has a 12 base single stranded overhang on the 5′ side of the Xa/LIC site and a 15-base single stranded overhang on the 3′ side of the Xa/LIC site. The plasmid is designed for ligation independent cloning, with N-terminal His and S-tags and an optional C-terminal His-tag (not used in this work). The Xa protease recognition site (IEGR) sits directly in front of the start codon of the gene of interest, such that the fusion protein tags can be removed.

Forward primer for E. coli nagA cloning
into pET30(Xa/LIC):
GGTATTGAGGGTCGCATGTATGCATTAACCCAGG
Reverse primer for E. coli nagA cloning
into pET30(Xa/LIC):
AGAGGAGAGTTAGAGCCTTATTGAGTTACGACCTCGT

Primers for the synthesis of the gene with appropriate restriction sequences for the pESCLeu vector 5‘of the gene’s ATG start codon and 3‘of each gene’s stop codon were designed for PCR amplification using E. coli DH10B genomic DNA as template. Forward primer for E. coli nagA cloning into pESCLeu with a BamHI restriction site:

GCGGATCCATGGCTGCATTAACCCAGG

Note: the fourth and fifth nucleotides of the open reading frame were changed from TA to GC to establish a Kozak sequence for cloning into S. cerevisiae. The translated sequence contains an alanine instead of tyrosine as the second amino acid.

Reverse primer for E. coli nagA cloning into pESCLeu with a XhoI restriction site:

CCGCTCGAGTTATTGAGTTACGACCTCGTTAC

Italics indicate the restriction sites while bold lettering indicates the start and stop codons.

Construction of nagApET30(Xa/LIC) and nagApESCLeu Vectors

The nagA gene was amplified by PCR using the primers described above with Expand DNA polymerase and the corresponding buffer containing magnesium, following the manufacturer's protocol. The thermocycler program used included a hot start at 96° C. for 2 min and 29 repetitions of the following steps: 94° C. for 30 sec, 66.5° C. for 1 min, and 72° C. for 1 min. After the 30 cycles the sample was incubated at 72° C. for 8 min and then stored at 4° C. The PCR products were purified from a 1% TAE-agarose gel (QIAQuick Gel Purification kit).

The PCR product for pET30(Xa/LIC) cloning was treated with T4 DNA polymerase following the manufacturer's recommended protocols for Ligation Independent Cloning (Novagen, Madison, Wis.). Briefly, approximately 0.2 pmol of purified PCR product was treated with 1 U T4 DNA polymerase, which has proofreading activity, in the presence of dGTP for 30 minutes at 22° C. The polymerase removes successive bases from the 3′ ends of the PCR product. When the polymerase encounters a guanine residue, the 5′ to 3′ polymerase activity of the enzyme counteracts the exonuclease activity to effectively prevent further excision. This creates single stranded overhangs that are compatible with the pET Xa/LIC vector. The polymerase was inactivated by incubating at 75° C. for 20 minutes. The vector and treated insert were annealed as recommended by Novagen. Approximately 0.02 pmol of treated insert and 0.01 pmol vector were incubated for 5 minutes at 22° C., 6.25 mM EDTA (final concentration) was added, and the incubation at 22° C. was repeated. One μL of the mixture was transformed into E. coli DH 10B ElectroMAX cells using the BioRad recommended procedure for transformation of E. coli cells with 0.1 cm micro-electroporation cuvettes. After recovery in SOC medium the transformation mixture was plated on LB plates containing kanamycin at 25 μg/mL. Plasmid DNA was isolated from liquid cultures [5 mL 2xYT medium+kanamycin (50 μg/mL) grown overnight at 37° C.] of colonies picked from the LB+kanamycin (25 μg/mL) plates and purified. The plasmids were then screened by restriction digestion and the sequences were verified by dideoxynucleotide chain-termination DNA sequencing. Plasmid with the correct sequence was subcloned into E. coli BL21(DE3) cells according to the manufacturer's protocol. After recovery in SOC medium the transformation mixture was plated on LB plates containing kanamycin at 25 μg/mL. Plasmid DNA was isolated from liquid cultures {5 mL 2xYT medium+kanamycin (50 μg/mL) grown overnight at 37° C.} of colonies picked from the LB+kanamycin (25 μg/mL) plates and purified. The plasmids were screened by restriction digestion to verify the gene insertion.

After restriction digestion of both the PCR product for cloning into pESCLeu and the pESCLeu vector with BamHI and XhoI, the ligation was carried out using the Rapid DNA Ligation Kit (Roche). The ligation mixture was desalted and then transformed into E. coli DH10B ElectroMAX cells using the BioRad recommended procedure for transformation of E. coli cells with 0.1 cm micro-electroporation cuvettes. After recovery in SOC medium the transformation mixture was plated on LB plates containing ampicillin at 100 μg/mL. Plasmid DNA was isolated from liquid cultures [5 mL 2xYT medium+ampicillin (100 μg/mL) grown overnight at 37° C.] of colonies picked from the LB+ampicillin (100 μg/mL) plates and purified. The plasmids were then screened by restriction digestion and the sequences were verified by dideoxynucleotide chain-termination DNA sequencing.

### Example 11

### S. Cerevisiae Strains with a Vector Carrying a nagA Gene and One Carrying the GFA1 Gene

The following example describes the procedure for the transformation of S. cerevisiae strains with a vector carrying a nagA gene and one carrying the GFA 1 gene. The resulting strains are expected to be suitable for high level production of glucosamine.

Competent cells of the S. cerevisiae strains listed below are prepared using an S.c. EasyComp™ Transformation Kit (Invitrogen Corp; Carlsbad, Calif.). Aliquots (50 μL) are frozen at −80° C. and thawed just prior to use.

S. cerevisiae Strains

| | |
|---|---|
| BY4742 | haploid parental strain (MATα, his3-Δ1, leu2-Δ0, lys2-Δ0, |
| | ura3-Δ0) |
| 12266 | PFK26 (YIL107C) deletion (6-phosphofructo-2-kinase) (haploid) |
| 16545 | PGM2 (YMR105C) deletion (phosphoglucomutase isozyme) |
| | (haploid) |
| 14977 | PGM1 (YKL127W) deletion (phosphoglucomutase minor form) |
| | (haploid) |
| 15893 | PFK1 (YGR240C) deletion (phosphofructokinase alpha subunit) |
| | (haploid) |
| 10791 | PFK2 (YMR205C) (phosphofructokinase beta subunit) (haploid) |
| BY4743 | diploid parental strain (MATa/α, his3-Δ1/his3-Δ1, leu2-Δ0/ |
| | leu2-Δ0, met15-Δ0/MET15⁺, LYS2⁺/lys2-Δ0, ura3-Δ0/ |
| | ura3-Δ0) |
| 20299 | PCM1 (YEL058W) deletion (NAcglucosamine-6-P mutase) |
| | (heterozygous diploid) |
| 23800 | QRI1, UAP1 (YDL103C) deletion (UDP-NAc-glucosamine |
| | pyrophosphorylase) (heterozygousdiploid) |
| 25635 | GNA1, PAT1 (YFL017C) deletion (glucosamine-phosphate |
| | N-acetyltransferase) (heterozygous diploid) |
| 20324 | PMI, PMI40 (YER003C) deletion (mannose-6-phosphate |
| | isomerase) (heterozygous diploid) |

Simultaneous transformations of the pESCHis vector containing the GFA1 gene (see Example 1) and the pESCLeu vector containing the nagA gene (see Example 10) into S. cerevisiae competent cells are also carried out using the S.c. EasyComp™ Transformation Kit. The vectors pESCHis and pESCLeu or GFA1pESCHis and nagApESCLeu are transformed into each strain. A 100 μL aliquot from each transformation reaction is spread on SC-His-Leu plates (medium recipes from Stratagene pESC manual). The plate medium of the strains with single gene deletions also contain 0.2 mg/mL geneticin. The plates are incubated for 2 days at 30° C. Colonies from each plate are used to inoculate 5 mL liquid cultures of SC-His-Leu medium. The cultures are incubated overnight at 30° C. and the cells are harvested by centrifugation, and plasmid DNA is isolated from the cells using a Zymoprep Yeast Plasmid Miniprep kit. After analysis of the isolated DNA by PCR, one isolate from each construct that generated the predicted PCR products is chosen for expression studies.

### Example 12

### Overexpression of the nagA Gene in E. coli BL21(DE3) and the Enzymatic Deacetylation of N-Acetylation in Fermentation Broth Samples

Induction of the nagA Gene

E. coli BL21(DE3) constructs carrying the pET30(Xa/LIC) plasmid with the E. coli nagA insert were grown in 5 mL 2xYT containing 50 μg/mL kanamycin overnight at 37° C. with shaking. One mL from each culture was transferred to 50 mL of LB medium containing 50 μg/mL kanamycin and the incubation was continued until the OD₆₀₀ was ˜0.5. The gene expression was induced by the addition of 0.1 mM IPTG and the incubation was continued at 30° C. for 4 h. The cells were harvested by centrifugation and stored at −80° C. until use.

Cell extracts were prepared from the 4 hour samples by suspending the cell pellets in 5 mL Novagen BugBuster™ reagent containing 1 μL benzonase nuclease (Novagen) and 5 μL of protease inhibitor cocktail III (Calbiochem) per gram of cell pellet, incubating at room temperature for 20 minutes with gentle shaking, and centrifuging at 21,000×g to remove cell debris. The supernatants (cell extracts) were analyzed for protein expression by one-dimensional gel electrophoresis using a Bio-Rad Protein 3 mini-gel system and pre-cast 4-15% gradient SDS-PAGE gels. The expressed protein constituted approximately 25-30% of the soluble protein fraction.

The nagA protein was purified using a His-Bind cartridge 900 following manufacturer's protocols (Novagen, Madison, Wis.). The eluent fractions were desalted on PD-10 (Amersham Biosciences, Piscataway, N.J.) columns and eluted in 50 mM Tris, pH 7.8. Purified proteins were analyzed by SDS-PAGE as described above.

Determination of Enzyme Activity with N-acetylglucosamine

The enzymatic deacetylation of N-acetylglucosamine was followed over time. In a total of 0.2 mL was mixed 0.1 mL of 500 mM N-acetylglucosamine, 0.02 mL of 0.5 M Tris-HCl, pH 7.8 and nagA protein (cell extract fraction; 3.5 mg/mL protein). The mixtures were incubated from 0 to 120 min at 37° C. To distinguish between glucosamine and N-acetylglucosamine using the Elson and Morgan method described in the Materials and Methods section, the product formation in the samples was measured with and without the acetylation step. The amount of glucosamine was calculated as the difference between the assay results with and without the acetylation step. The results are summarized in Table 12. After 120 min approximately 74% of the substrate N-acetylglusosamine was converted to glucosamine.

TABLE 12
Time course of the deacetylation of N-acetylglucosamine
by E. coli N-acetylglucosamine-6-phosphate deacetylase

| Time (min) | [Glucosamine]; mM |
|---|---|
| 0 | 0.0 |
| 15 | 126.3 |
| 30 | 136.3 |
| 60 | 161.3 |
| 120 | 185.0 |

Enzymatic Deacetylation of N-acetylglucosamine in Fermentation Broth Samples
Fermentation Broth Sample Preparation

S. cerevisiae strains 16545 and BY4742 carrying the pESCHis plasmid with or without the GFA1 insert were grown in 5 mL SC-His containing 2% glucose overnight at 30° C. with shaking. One mL from each culture was transferred to 5 mL of SC-His medium containing 1% raffinose and 1% glucose and the incubation was continued for 10 h. The medium of the strain 16545 also contained 0.2 mg/mL geneticin. The OD₆₀₀ of each culture was determined and the amount of culture necessary to obtain an OD₆₀₀ of 0.4 in 5 mL of SC-His containing 1% galactose and 1% raffinose (induction medium) was calculated. The calculated volume of cells was centrifuged at 1500×g for 10 min at 4° C. and the pellet was resuspended in 5 mL induction medium. Each construct was grown at 30° C. with shaking at 250 rpm from 0 to 72 h. Aliquots of fermentation broth at several time points after induction were removed and were centrifuged to remove the cells. The supernatants were frozen at −80° C.

Determination of the N-acetylglucosamine Deacetylation in Broth Samples

The enzymatic deacetylation of N-acetylglucosamine was carried out in the fermentation broth samples withdrawn at 72 h after induction. In a total of 0.2 mL was mixed 0.1 mL of N-acetylglucosamine sample, 0.02 mL 0.5 M Tris-HCl, pH 7.8 and nagA protein (cell extract fraction; 3.5 mg/mL protein). The mixtures were incubated for 60 min at 37° C. To distinguish between glucosamine and N-acetylglucosamine using the Elson and Morgan method described in the Materials and Methods section, the product formation in the samples was measured with and without the acetylation step. The results of the assays of the fermentation broth samples after enzymatic deacetylation are summarized in Table 13.

TABLE 13
Deacetylation of N-acetylglucosamine in fermentation broth samples
by E. coli N-acetylglucosamine-6-phosphate deacetylase

| | | [Product]; mM | |
|---|---|---|---|
| Strain | Vector | +Acetylation Step | −Acetylation Step |
| BY4742 | pESCHis | 0.1 | 0 |
| BY4742 | GFA1pESCHis | 1.15 | 0 |
| 16545 | pESCHis | 0.05 | 0 |
| 16545 | GFA1pESCHis | 2.15 | 0 |

### Example 13

### Simultaneous Overexpression of the GFA1 And nagA Genes in S. Cerevisiae Strains and Accumulation Of Glucosamine and/or N-Acetylglucosamine in the Fermentation Broth

Induction of the GFA1 Gene

S. cerevisiae strains carrying the pESCHis plasmid with or without the GFA1 insert and the pESCLeu plasmid with or without the nagA insert are grown in 5 mL SC-His-Leu containing 2% glucose overnight at 30° C. with shaking. One mL from each culture is transferred to 5 mL of SC-His-Leu medium containing 1% raffinose and 1% glucose and the incubation is continued for 10 h. The medium of the strains with single gene deletions also contains 0.2 mg/mL geneticin. The OD₆₀₀ of each culture is determined and the amount of culture necessary to obtain an OD₆₀₀ of 0.16 to 0.4 in 100 mL of SC-His-Leu containing 1% galactose and 1% raffinose (induction medium) is calculated. The calculated volume of cells is centrifuged at 1500×g for 10 min at 4° C. and the pellet is resuspended in 100 mL induction medium. Each construct is grown at 30° C. with shaking at 250 rpm from 0 to 90 h.

Determination of Glucosamine and N-Acetylglucosamine Formation

At several time points after induction aliquots of fermentation broth are removed, the OD₆₀₀ is measured, and then the aliquots are centrifuged to remove the cells and the supernatants are frozen at −80° C. The cell pellet fractions from the aliquots harvested are also frozen at −80° C.

Product formation is determined in the thawed samples using the methods described in the Materials and Methods section above. To distinguish between glucosamine and N-acetylglucosamine using the Elson and Morgan method, the assays are carried out with and without the acetylation step.

1. A method for producing an amino sugar in diploid yeast selected from the group consisting of N-acetylglucosamine, glucosamine, or a combination thereof comprising culturing a diploid yeast in a culture medium and recovering N-acetylglucosamine, glucosamine, or a combination thereof,
wherein said yeast comprises an exogenous nucleic acid sequence encoding glucosamine-6-phosphate synthase operably linked to a promoter.

2. A method for producing glucosamine in diploid yeast comprising culturing a diploid yeast in a culture medium, performing deacetylation, and recovering glucosamine,
wherein said yeast comprises an exogenous nucleic acid sequence encoding glucosamine-6-phosphate synthase operably linked to a promoter.

3. A method for producing an amino sugar in diploid yeast selected from the group consisting of N-acetylglucosamine, glucosamine, or a combination thereof comprising culturing a diploid yeast in a culture medium and recovering N-acetylglucosamine, glucosamine, or a combination thereof,
wherein
(a) said yeast comprises an exogenous nucleic acid sequence encoding glucosamine-6-phosphate synthase operably linked to a promoter, and
(b) the pH of the culture medium is equal to or less than pH 5.0.

4. The method of claim 1, 2 or 3, wherein said nucleic acid sequence encoding glucosamine-6-phosphate synthase comprises a genetic modification which reduces feedback inhibition of said glucosamine-6-phosphate synthase.

5. The method of claim 2, wherein said deacetylation comprises contacting said culture medium with acid or an enzyme.

6. The method of claim 2, wherein prior to performing deacetylation said yeast is separated from said culture medium and deacetylation is performed on the culture medium.

7. The method of claim 5, wherein said enzyme is N-acetylglucosamine-6-phosphate deacetylase.

8. The method of claim 7, where said N-acetylglucosamine-6-phosphate deacetylase deacetylase is from the division Gammaproteobacteria.

9. The method of claim 7, where said N-acetylglucosamine-6-phosphate deacetylase or glucosamine-6-phosphate deacetylase is from Escherichia coli.

10. The method of claim 5, wherein said acid is selected from the group consisting of hydrochloric acid, sulfuric acid, nitric acid, nitrous acid, perchloric acid and phosphoric acid.

11. The method of claim 1, 2 or 3 wherein said nucleic acid sequence encoding glucosamine-6-phosphate synthase encodes yeast glucosamine-6-phosphate synthase.

12. The method of claim 11 wherein said yeast is Saccharomyces cerevisiae.

13. The method of claim 11, wherein said yeast glucosamine-6-phosphate synthase gene is GFA1.

14. The method of claim 1, 2 or 3 wherein said yeast further comprises one or more genetic modifications that minimize degradation of glucosamine-6-phosphate by said yeast.

15. The method of claim 14 wherein said one or more genetic modifications comprises disruption of a nucleic acid sequence encoding a peptide selected from the group consisting of 6-phosphofructo-2-kinase, phosphoglucomutase, phosphofructokinase alpha subunit, phosphofructokinase beta subunit, N-acetylglucosamine-6-phosphate mutase, UDP N-acetylglucosamine-6-phosphate pyrophosphorylase, glucosamine-6-phosphate N-acetyltransferase, mannose-6-phosphate isomerase, hexokinase I and chitin synthase.

16. The method of claim 14, wherein said yeast is heterozygous diploid and said one or more genetic modifications comprises disruption of a nucleic acid sequence encoding a peptide selected from the group consisting of N-acetylglucosamine-6-phosphate mutase, UDP N-acetylglucosamine-6-phosphate pyrophosphorylase, glucosamine-6-phosphate N-acetyltransferase, mannose-6-phosphate isomerase, hexokinase I and chitin synthase.

17. The method of claim 14, wherein said yeast is homozygous diploid and said one or more genetic modifications comprises disruption of a nucleic acid sequence encoding a peptide selected from the group consisting of 6-phosphofructo-2-kinase, phosphoglucomutase, phosphofructokinase alpha subunit, phosphofructokinase beta subunit, hexokinase I and chitin synthase.

18. The method of claim 14 wherein said one or more genetic modifications comprises disruption of a nucleic acid sequence encoding glucosamine-phosphate N-acetyltransferase in said yeast.

19. The method of claim 14 wherein said one or more genetic modifications comprises disruption of a nucleic acid sequence encoding phosphoglucomutase.

20. The method of claim 14 wherein said one or more genetic modifications comprises disruption of a nucleic acid sequence encoding UDP N-acetylglucosamine-6-phosphate pyrophosphorylase.

21. The method of claim 1, 2, or 3 wherein said yeast is a MATalpha strain comprising:
(a) an exogenous nucleic acid sequence encoding alpha-factor pheromone receptor; and
(b) a genetic modification comprising disruption of a nucleic acid sequence encoding a-factor pheromone receptor.

22. The method of claim 2 wherein said glucosamine is recovered by evaporative crystallization.

23. The method of claim 1, 2 or 3, wherein the pH of said culture medium is equal to or less than pH 5.0.

24. A genetically modified diploid yeast comprising
(a) an exogenous nucleic acid sequence encoding glucosamine-6-phosphate synthase; and
(b) one or more genetic modifications comprising disruption of a nucleic acid sequence encoding a peptide selected from the group consisting of 6-phosphofructo-2-kinase, phosphoglucomutase, phosphofructokinase alpha subunit, phosphofructokinase beta subunit, N-acetylglucosamine-6-phosphate mutase, UDP N-acetylglucosamine-6-phosphate pyrophosphorylase, glucosamine-6-phosphate N-acetyltransferase, mannose-6-phosphate isomerase, hexokinase I and chitin synthase.

25. The method of claim 1 or 3, wherein said N-acetylglucosamine, glucosamine or combination thereof further comprises one or more carbohydrates.

26. The method of claim 2, wherein said glucosamine further comprises one or more carbohydrates.

27. The method of claim 3 wherein said culture medium lacks an antimicrobial agent.

28. The method of claim 1 wherein said amino sugar is N-acetylglucosamine.

29. A genetically modified yeast of claim 24 in a culture medium of pH less than 5 containing an amino sugar selected from the group consisting of N-acetylglucosamine, glucosamine, or a combination thereof [symbol]


SEQUENCE LISTING (35 sequences)

SEQ ID NO: 1 - DNA, 2154 bp, Saccharomyces cerevisiae
atgtgtggtatctttggttactgcaattatctagtggaaagatccagaggagaaattatcgacaccttagtggatggtttacaaagattagaatatagaggctatgattccaccggtattgctatcgatggtgacgaagctgattctactttcatctataagcaaatcggtaaagtgagtgctttgaaagaggagattactaagcaaaatccgaacagagacgttacttttgtctctcattgtggtattgcgcatactagatgggctactcacggtcgaccagaacaagttaactgtcaccctcaaagatctgacccagaagaccaatttgtggtcgttcataatggtatcatcacaaattttagagaactgaagactcttttaattaacaaaggttataaattcgaaagtgataccgataccgagtgtattgctaaactatatttgcatttatacaatacaaatttacaaaatgggcatgacttagatttccacgaattaaccaagctagttcttttagaactagaaggttcatacgggttattatgtaaatcttgtcactatcctaatgaggttatcgccactagaaaagggtcccctttactgattggtgtcaaatctgaaaaaaaactaaaagtcgacttcgtggatgtggaatttcccgaagaaaacgctggtcaaccggaaattccattgaaatctaacaacaaatcatttggcttgggcccaaagaaagctcgtgaatttgaagctggttcccaaaatgccaatttactaccaattgccgccaatgaatttaacttgagacattctcaatccagggctttcctatcagaagatggatctccaacaccggtggaattttttgtttcttcggatgcggcatctgttgttaaacataccaagaaggtgctatttttagaagatgacgatttggctcatatttacgatggtgagttacatattcatagatctagaagagaagtaggcgcatcaatgacaaggtccattcaaactttagagatggagttagctcagatcatgaagggcccttacgaccattttatgcaaaaggaaatctatgagcaaccagaatctactttcaatactatgagaggtagaatcgactatgaaaataataaagtgatattgggtggtttaaaggcatggttaccagttgtcagaagagcacggagactgatcatgatcgcatgcggtacttcttatcattcatgtttggctactcgtgctatcttcgaagaattatcagatatcccagttagtgtggaattagcgtctgactttctggacagaaaatgccctgtcttcagagacgatgtatgcgtgtttgtttcacaaagtggtgaaactgcggataccatgctggctctaaattattgtttagaaagaggagccttaactgtcggaattgttaacagtgttggttcttctatctctcgtgtcacccactgtggtgttcatattaacgctggtcctgaaattggtgttgcctctacaaaagcttatacttcccagtatattgccttagtgatgtttgctctatcgctgtcagatgaccgtgtatcgaaaatagacagaagaattgaaatcattcaaggcttgaagttaatcccgggccaaattaagcaggtattaaagctggaaccaagaataaaaaagctctgtgcgactgaattaaaggatcaaaaatctctattgttattgggtagaggttaccaatttgctgctgctctggaaggtgctttgaagatcaaagaaatttcttatatgcattctgaaggtgttttggcaggtgagttgaagcacggtgtcttggccttggtggacgaaaacttgccaatcattgcttttggtaccagagactctctattccctaaagtagtttcctctattgagcaagttactgcaagaaagggccatccaattattatttgtaacgaaaatgatgaagtgtgggcgcaaaaatctaaatcaatcgacctgcaaaccttagaagttccacaaactgttgattgtttacaaggtctaattaatattattccattacaactaatgtcatattggttggctgttaataaagggattgatgttgattttccaagaaacttggctaaatctgttaccgtcgaataa

SEQ ID NO: 2 - protein, 717 aa, Saccharomyces cerevisiae
MCGIFGYCNYLVERSRGEIIDTLVDGLQRLEYRGYDSTGIAIDGDEADSTFIYKQIGKVSALKEEITKQNPNRDVTFVSHCGIAHTRWATHGRPEQVNCHPQRSDPEDQFVVVHNGIITNFRELKTLLINKGYKFESDTDTECIAKLYLHLYNTNLQNGHDLDFHELTKLVLLELEGSYGLLCKSCHYPNEVIATRKGSPLLIGVKSEKKLKVDFVDVEFPEENAGQPEIPLKSNNKSFGLGPKKAREFEAGSQNANLLPIAANEFNLRHSQSRAFLSEDGSPTPVEFFVSSDAASVVKHTKKVLFLEDDDLAHIYDGELHIHRSRREVGASMTRSIQTLEMELAQIMKGPYDHFMQKEIYEQPESTFNTMRGRIDYENNKVILGGLKAWLPVVRRARRLIMIACGTSYHSCLATRAIFEELSDIPVSVELASDFLDRKCPVFRDDVCVFVSQSGETADTMLALNYCLERGALTVGIVNSVGSSISRVTHCGVHINAGPEIGVASTKAYTSQYIALVMFALSLSDDRVSKIDRRIEIIQGLKLIPGQIKQVLKLEPRIKKLCATELKDQKSLLLLGRGYQFAAALEGALKIKEISYMHSEGVLAGELKHGVLALVDENLPIIAFGTRDSLFPKVVSSIEQVTARKGHPIIICNENDEVWAQKSKSIDLQTLEVPQTVDCLQGLINIIPLQLMSYWLAVNKGIDVDFPRNLAKSVTVE

SEQ ID NO: 3 - DNA, 1149 bp, Escherichia coli
atgtatgcattaacccagggccggatctttaccggccacgaatttcttgatgaccacgcggttgttatcgctgatggcctgattaaaagcgtctgtccggtagcggaactgccgccagagatcgaacaacgttcactgaacggggccattctctcccccggttttatcgatgtgcagttaaacggctgcggcggcgtacagtttaacgacaccgctgaagcggtcagcgtggaaacgctggaaatcatgcagaaagccaatgagaaatcaggctgtactaactatctgccgacgcttatcaccaccagcgatgagctgatgaaacagggcgtgcgcgttatgcgcgagtacctggcaaaacatccgaatcaggcgttaggtctgcatctggaaggtccgtggctgaatctggtaaaaaaaggcacccataatccgaattttgtgcgtaagcctgatgccgcgctggtcgatttcctgtgtgaaaacgccgacgtcattaccaaagtgaccctggcaccggaaatggttcctgcggaagtcatcagcaaactggcaaatgccgggattgtggtttctgccggtcactccaacgcgacgttgaaagaagcaaaagccggtttccgcgcggggattacctttgccacccatctgtacaacgcgatgccgtatattaccggtcgtgaacctggcctggcgggcgcgatcctcgacgaagctgacatttattgcggtattattgctgatggcctgcatgttgattacgccaacattcgcaacgctaaacgtctgaaaggcgacaaactgtgtctggttactgacgccaccgcgccagcaggtgccaacattgaacagttcatttttgcgggtaaaacaatatactaccgtaacggactttgtgtggatgagaacggtacgttaagcggttcatccttaaccatgattgaaggcgtgcgtaatctggtcgaacattgcggtatcgcactggatgaagtgctacgtatggcgacgctctatccggcgcgtgcgattggcgttgagaaacgtctcggcacactcgccgcaggtaaagtagccaacctgactgcattcacacctgattttaaaatcaccaagaccatcgttaacggtaacgaggtcgtaactcaataa

SEQ ID NO: 4 - protein, 382 aa, Escherichia coli
MYALTQGRIFTGHEFLDDHAVVIADGLIKSVCPVAELPPEIEQRSLNGAILSPGFIDVQLNGCGGVQFNDTAEAVSVETLEIMQKANEKSGCTNYLPTLITTSDELMKQGVRVMREYLAKHPNQALGLHLEGPWLNLVKKGTHNPNFVRKPDAALVDFLCENADVITKVTLAPEMVPAEVISKLANAGIVVSAGHSNATLKEAKAGFRAGITFATHLYNAMPYITGREPGLAGAILDEADIYCGIIADGLHVDYANIRNAKRLKGDKLCLVTDATAPAGANIEQFIFAGKTIYYRNGLCVDENGTLSGSSLTMIEGVRNLVEHCGIALDEVLRMATLYPARAIGVEKRLGTLAAGKVANLTAFTPDFKITKTIVNGNEVVTQ

SEQ ID NO: 5 - DNA, 1413 bp, Saccharomyces cerevisiae
atgtcatacaagtcagcaataatagggctttgtttgctagctgtgatactattagctccccctttagcatggcattcacataccaagaatattccagcaatcattttgataacatggcttcttacaatgaatttaacgtgtattgtagatgcggcaatatggagtgacgacgatttcctcacgagatgggatggtaaaggttggtgtgatattgtcatcaagttgcaagttggtgcgaatattggcatatcatgtgccgttaccaacatcatttacaacttgcatacaattttgaaggcagatagtgttttaccggatctttcatcatggacgaaaatcgtcaaggaccttgtgattagcttgttcacacctgtcatggtcatgggattttcatatctgttacaagtatttagatatggtatcgctcgctacaacggttgccaaaacttattgtctccgacgtggattaccactgttttgtataccatgtggatgcttatatggtcatttgtgggcgctgtttatgccaccttagtactgttcgtgttttataaaaaacgcaaggacgttagggatattttacactgtaccaattcaggtttaaacctgacaaggttcgcaaggctgttaatattctgtttcattattattttagtcatgttccctttttctgtttacacctttgttcaagatttacagcaggtagaaggacactatacttttaaaaatacccattccagcactatctggaataccattattaaatttgaccctggcagaccaatttataatatatggctttatgttttgatgtcttacctagtatttctaatctttggcttaggttctgatgctttgcatatgtactctaaattcctgcgttccatcaaactaggatttgtacttgacatgtggaaaagattcattgataagaataaggaaaaacgagtaggcatattgctaaacaagctgtcctcacgcaaagagagtcgtaacccattttctacagactctgagaactatatctccacgtgtacagaaaactattctccctgtgtaggtacaccaatatcacaagcgcatttctatgtcgactataggattccagatgatcctagaaaatctcaaaataaaagcaaaaaatatttgtttgctgataaagaaacagatgatattcttgatgaaatagacctaaaagaaagtaggcacatcccttacgtcacgcaaggacagagctttgacgacgaaatatcacttggaggattctcaaaagttactctcgattattcagaaaagcttcataattctgcaagctccaattttgaaggggaaagtctttgctactctccagcttcaaaagaagagaattcaagctcaaacgaacatagttcagaaaatactgcaggcccttaa

SEQ ID NO: 6 - protein, 470 aa, Saccharomyces cerevisiae
MSYKSAIIGLCLLAVILLAPPLAWHSHTKNIPAIILITWLLTMNLTCIVDAAIWSDDDFLTRWDGKGWCDIVIKLQVGANIGISCAVTNIIYNLHTILKADSVLPDLSSWTKIVKDLVISLFTPVMVMGFSYLLQVFRYGIARYNGCQNLLSPTWITTVLYTMWMLIWSFVGAVYATLVLFVFYKKRKDVRDILHCTNSGLNLTRFARLLIFCFIIILVMFPFSVYTFVQDLQQVEGHYTFKNTHSSTIWNTIIKFDPGRPIYNIWLYVLMSYLVFLIFGLGSDALHMYSKFLRSIKLGFVLDMWKRFIDKNKEKRVGILLNKLSSRKESRNPFSTDSENYISTCTENYSPCVGTPISQAHFYVDYRIPDDPRKSQNKSKKYLFADKETDDILDEIDLKESRHIPYVTQGQSFDDEISLGGFSKVTLDYSEKLHNSASSNFEGESLCYSPASKEENSSSNEHSSENTAGP

SEQ ID NO: 7 - DNA, 29 bp, Artificial Sequence
  Description of Artificial Sequence Synthetic Primer
cgcggatccagaatgtgtggtatctttgg

SEQ ID NO: 8 - DNA, 33 bp, Artificial Sequence
  Description of Artificial Sequence Synthetic Primer
ccgctcgagttattcgacggtaacagatttagc

SEQ ID NO: 9 - DNA, 32 bp, Artificial Sequence
  Description of Artificial Sequence Synthetic Primer
ggactagtatgtgtggtatctttggttactgc

SEQ ID NO: 10 - DNA, 33 bp, Artificial Sequence
  Description of Artificial Sequence Synthetic Primer
ccggagctcttattcgacggtaacagatttagc

SEQ ID NO: 11 - DNA, 29 bp, Artificial Sequence
  Description of Artificial Sequence Synthetic Primer
ccagaatgtgtggtacctttggttactgc

SEQ ID NO: 12 - DNA, 29 bp, Artificial Sequence
  Description of Artificial Sequence Synthetic Primer
gctatcgatggtaccgaagctgattctac

SEQ ID NO: 13 - DNA, 32 bp, Artificial Sequence
  Description of Artificial Sequence Synthetic Primer
gaagggcccttactgccattttatgcaaaagg

SEQ ID NO: 14 - DNA, 33 bp, Artificial Sequence
  Description of Artificial Sequence Synthetic Primer
caatactatgagaggtagaaccgactatgaaaa

SEQ ID NO: 15 - DNA, 35 bp, Artificial Sequence
  Description of Artificial Sequence Synthetic Primer
gcaggtattaccgctggaaccaagaataaaaaagc

SEQ ID NO: 16 - DNA, 38 bp, Artificial Sequence
  Description of Artificial Sequence Synthetic Primer
ggatcaaaaatctctaccgttattgggcagaggttacc

SEQ ID NO: 17 - DNA, 33 bp, Artificial Sequence
  Description of Artificial Sequence Synthetic Primer
ctctattgttattgagtagaggttaccaatttg

SEQ ID NO: 18 - DNA, 38 bp, Artificial Sequence
  Description of Artificial Sequence Synthetic Primer
ggatcaaaaatctctaccgttattgagtagaggttacc

SEQ ID NO: 19 - DNA, 25 bp, Artificial Sequence
  Description of Artificial Sequence Synthetic Primer
gaaaaaaccccggatccagaatgtg

SEQ ID NO: 20 - DNA, 27 bp, Artificial Sequence
  Description of Artificial Sequence Synthetic Primer
gcggtaccaagcttactcgagttattc

SEQ ID NO: 21 - DNA, 60 bp, Artificial Sequence
  Description of Artificial Sequence Synthetic Primer
caaagaaacgcagatataaaggagaacaccagatcgaactagcttcgtacgctgcaggtc

SEQ ID NO: 22 - DNA, 58 bp, Artificial Sequence
  Description of Artificial Sequence Synthetic Primer
tacatgttaaaagtgcttcataagtagctcaaacagccgcataggccactagtggatc

SEQ ID NO: 23 - DNA, 20 bp, Artificial Sequence
  Description of Artificial Sequence Synthetic Primer
cactaaccagcaggctaaca

SEQ ID NO: 24 - DNA, 18 bp, Artificial Sequence
  Description of Artificial Sequence Synthetic Primer
ttcgcctcgacatcatct

SEQ ID NO: 25 - DNA, 37 bp, Artificial Sequence
  Description of Artificial Sequence Synthetic Primer
cgcggatccagaatgtcatacaagtcagcaataatag

SEQ ID NO: 26 - DNA, 29 bp, Artificial Sequence
  Description of Artificial Sequence Synthetic Primer
aagctcgagttaagggcctgcagtatttt

SEQ ID NO: 27 - DNA, 34 bp, Artificial Sequence
  Description of Artificial Sequence Synthetic Primer
ggactagtatgtcatacaagtcagcaataatagg

SEQ ID NO: 28 - DNA, 31 bp, Artificial Sequence
  Description of Artificial Sequence Synthetic Primer
aatgagctcttaagggcctgcagtattttct

SEQ ID NO: 29 - DNA, 60 bp, Artificial Sequence
  Description of Artificial Sequence Synthetic Primer
gcttacccgatggattttatataaggcgaatggaagagggagcttcgtacgctgcaggtc

SEQ ID NO: 30 - DNA, 62 bp, Artificial Sequence
  Description of Artificial Sequence Synthetic Primer
aatggagataaatggtgaagaccctgccaataaccaccagccgcataggccactagtggatc

SEQ ID NO: 31 - DNA, 20 bp, Artificial Sequence
  Description of Artificial Sequence Synthetic Primer
cgcggagacttctcgccaat

SEQ ID NO: 32 - DNA, 34 bp, Artificial Sequence
  Description of Artificial Sequence Synthetic Primer
ggtattgagggtcgcatgtatgcattaacccagg

SEQ ID NO: 33 - DNA, 37 bp, Artificial Sequence
  Description of Artificial Sequence Synthetic Primer
agaggagagttagagccttattgagttacgacctcgt

SEQ ID NO: 34 - DNA, 27 bp, Artificial Sequence
  Description of Artificial Sequence Synthetic Primer
gcggatccatggctgcattaacccagg

SEQ ID NO: 35 - DNA, 32 bp, Artificial Sequence
  Description of Artificial Sequence Synthetic Primer
ccgctcgagttattgagttacgacctcgttac